(19) United States
(12) Patent Application Publication
(10) Pub. No.: US 2014/0121703 A1
(43) Pub. Date: May 1, 2014

(54) POLYAXIAL BONE ANCHOR WITH POP-ON MULTI-THREAD SHANK, SOME WITH DIAMETRIC INTERFERENCE FIT INSERTS

(71) Applicants: Roger P. Jackson, Prairie Village, KS (US); James L Surber, Kansas City, KS (US)

(72) Inventors: Roger P. Jackson, Prairie Village, KS (US); James L Surber, Kansas City, KS (US)

(21) Appl. No.: 14/061,393

(22) Filed: Oct. 23, 2013

Related U.S. Application Data
(60) Provisional application No. 61/795,984, filed on Oct. 31, 2012.
(60) Provisional application No. 61/851,223, filed on Mar. 4, 2013.

(51) Int. Cl. A61B 17/70

(52) U.S. Cl. CPC A61B 17/7032
USPC 606/246

(57) ABSTRACT

Polyaxial and uni-planar bone screw assemblies include a multi-threaded shank body having an integral upper portion receivable in a one piece receiver, the receiver having an upper channel for receiving a longitudinal connecting member and a lower cavity cooperating with a lower opening. A compression insert and open retaining ring located in the receiver may be made of cobalt-chrome and the compression insert may provide a diametric interference fit with the receiver. Receivers or inserts may include resilient arm portions. The open ring cooperates with the shank to provide for pop- or snap-on assembly of the shank with the receiver either prior to or after implantation of the shank into a vertebra.

### CROSS-REFERENCE TO RELATED APPLICATIONS

This application claims the benefit of U.S. Provisional Patent Application Ser. No. 61/795,984 filed Oct. 31, 2012 and U.S. Provisional Patent Application Ser. No. 61/851,223 filed Mar. 4, 2013, both of which are incorporated by reference herein.

### BACKGROUND OF THE INVENTION

The present invention is directed to bone anchors for use in bone surgery, particularly spinal surgery and particularly to polyaxial and uni-planar bone screws with compression or pressure inserts and expansion lock split retainers to snap over, capture and retain the bone screw shank head in the receiver member assembly and later fix the bone screw shank with respect to the receiver assembly.

Bone screws are utilized in many types of spinal surgery in order to secure various implants to vertebrae along the spinal column for the purpose of stabilizing and/or adjusting spinal alignment. Although both closed-ended and open-ended bone screws are known, open-ended screws are particularly well suited for connections to rods and connector arms, because such rods or arms do not need to be passed through a closed bore, but rather can be laid or urged into an open channel within a receiver or head of such a screw. Generally, the screws must be inserted into the bone as an integral unit along with the head, or as a preassembled unit in the form of a shank and pivotal receiver, such as a polyaxial bone screw assembly.

Typical open-ended bone screws include a threaded shank with a pair of parallel projecting branches or arms which form a yoke with a U-shaped slot or channel to receive a rod. Hooks and other types of connectors, as are used in spinal fixation techniques, may also include similar open ends for receiving rods or portions of other fixation and stabilization structure.

A common approach for providing vertebral column support is to implant bone screws into certain bones which then in turn support a longitudinal structure such as a rod, or are supported by such a rod. Bone screws of this type may have a fixed head or receiver relative to a shank thereof, or may be of a polyaxial screw nature. In the fixed bone screws, the rod receiver head cannot be moved relative to the shank and the rod must be favorably positioned in order for it to be placed within the receiver head. This is sometimes very difficult or impossible to do. Therefore, polyaxial bone screws are commonly preferred. Open-ended polyaxial bone screws typically allow for a loose or floppy rotation of the head or receiver about the shank until a desired rotational position of the receiver is achieved by fixing such position relative to the shank during a final stage of a medical procedure when a rod or other longitudinal connecting member is inserted into the receiver, followed by a locking screw or other closure. This loose or floppy feature can be, in some cases, undesirable, but may not be that detrimental in others. Also, it is often desirable to insert the bone screw shank separate from the receiver or head due to its bulk which can get in the way of what the surgeon needs to do. Such screws that allow for this capability are sometimes referred to as modular polyaxial screws.

### SUMMARY OF THE INVENTION

An embodiment of a polyaxial bone screw assembly according to the invention includes a shank having an integral upper portion or head that has at least one curved, radiused or spherical surface and a body for fixation to a bone; a separate receiver defining an upper open channel, a central bore, a lower cavity and a lower opening; a resilient, expansion locking split retainer for capturing the shank head in the receiver lower cavity and an insert having a friction fit portion, the shank head being frictionally engaged with, but still movable in a non-floppy manner, if desired, with respect to the friction fit insert prior to locking of the shank into a desired configuration. The shank is finally locked into a fixed position relative to the receiver by frictional engagement between the shank head and the insert and the shank head and one or more inner edges or surfaces of the split ring-like retainer due to a downward force placed on the compression insert by a tool or by a closure top pressing on a rod, or other longitudinal connecting member, captured within the receiver bore and channel. In certain illustrated embodiments, retainers and compression inserts are made from a harder material than a material or materials from which the receivers and shanks are made. In other embodiments, a harder shank may engage a compression insert made from of a less hard material. In an illustrated embodiment, the retainer and the insert are made from a cobalt-chrome alloy while the receiver and shank are made from a titanium alloy. In another embodiment the shank is made from cobalt chrome and the insert is made from a softer material. Receivers and/or inserts may include resilient arm portions. Also, in the illustrated embodiments, retainers and compression inserts are downloaded into the receiver, but uploaded embodiments are also foreseen. The shank head can be positioned into the receiver lower cavity at the lower opening thereof prior to or after insertion of the shank into bone. An illustrated compression insert includes diametric surfaces that cooperate with the receiver to result in a press fit of the insert against the receiver that provides a lock and release feature for independent locking of the polyaxial mechanism so the screw can be used like a fixed monoaxial screw. Such a locking frictional fit is thus along a run of the rod or other longitudinal connecting member, advantageously minimizing outward splay of the receiver arms. Also, the shank and other components of the assembly can be cannulated for minimally invasive surgery applications. Furthermore, an illustrated shank body has a lower segment or portion with a bottom or distal end having two starts resulting in two thread forms advancing upwardly to a mid-portion of the shank body wherein an upper segment has a three-start thread form wound thereon. As compared to prior art shanks that may, for example, interleave an additional thread at a mid section of a shank or transition a two start form into a four start or threaded form by interleaving a thread form between each existing form, the illustrated shank is preferably manufactured in two sections, with two separate or distinct forms and a transition area therebetween where the forms connect and morph into one another. A minor diameter defining the forms remains substantially constant along an entire length of the shank. Although a two start helical thread form/to three start helical thread form shank is illustrated, other forms are anticipated, for example, a three start helical form/to five start helical form shank body.

The expansion-only retainer ring base portion in an embodiment of the present invention is positioned entirely below the shank head hemisphere in the receiver and can be a stronger, harder, more substantial structure to resist larger pull out forces on the assembly, such as a structure made from cobalt chrome. Furthermore, to provide greater resiliency, the illustrated embodiment includes spaced grooves or notches running between top and bottom surfaces of the retainer. The retainer ring base can also be better supported on a planar shelf of the receiver having one or more horizontal loading surfaces located near the lower opening in the bottom of the receiver. Once assembled it cannot be disassembled.

A pre-assembled receiver, compression insert and split retainer may be “pushed-on”, “snapped-on” or “popped-on” to the shank head prior to or after implantation of the shank into a vertebra. Such a “snapping on” procedure includes the steps of uploading the shank head into the receiver lower opening, the shank head pressing against the base portion of the split retainer ring, pushing the ring up against the compression insert and expanding the resilient open retainer out into an expansion portion or chamber of the receiver cavity followed by an elastic return of the retainer back to a nominal or near nominal shape thereof after the hemisphere of the shank head or upper portion passes through the retainer. In some embodiments, sometimes with the aid of tooling, the shank head enters into a friction fit engagement with a lower collet-like portion of the insert. Final fixation occurs as a result of a locking expansion-type of contact between the shank head and the split retainer and an expansion-type of non-tapered locking engagement between the retainer ring and a lower receiver portion partially defining the receiver cavity. The retainer can expand more in an upper portion or expansion chamber of the receiver cavity to allow the shank head to pass through, but has restricted expansion to retain the shank head when the retainer ring is against the surfaces defining the lower portion of the receiver cavity. The shank head is forced down against the retainer ring during final locking by the compression insert. In some embodiments, when the polyaxial mechanism is locked, opposing outer surfaces of the pressure or compression insert are forced or wedged against surfaces of the receiver resulting in a press fit or interference locking engagement, allowing for adjustment or removal of the rod or other connecting member without loss of a desired angular relationship between the shank and the receiver. This independent locking feature allows the polyaxial screw to function like a fixed monoaxial screw.

The lower pressure insert may also be configured to be independently locked by a tool or instrument, thereby allowing the pop-on polyaxial screw to be distracted, compressed and/or rotated along and around the rod to provide for improved spinal correction techniques. Such a tool may engage the insert through apertures in the receiver to force or wedge the insert down into a locked position within the receiver. With the tool still in place and the correction maintained, the rod may then be locked within the receiver channel by a closure top followed by removal of the tool. This process may involve multiple screws all being manipulated simultaneously with multiple tools to achieve the desired correction.

A pop-on uni-planar bone screw assembly according to an embodiment of the invention includes a lower pressure insert and in some embodiments, an open retainer having planar surfaces cooperating with planar surfaces of a shank head to result in a shank that pivots only along a direction of the rod. The shank head typically includes opposed planar sides that cooperate with opposed planar surfaces of at least one of the compression insert and the retainer, limiting pivot to a single plane.

Objects of the invention further include providing apparatus and methods that are easy to use and especially adapted for the intended use thereof and wherein the tools are comparatively inexpensive to produce. Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is an exploded and partial front elevational view of a polyaxial bone screw assembly according to an embodiment of the present invention with portions broken away to show the detail thereof and including a shank, a receiver, an open retainer and a lower compression insert having a compressive friction fit lower collet.

FIG. 2 is an enlarged perspective view of the shank of FIG. 1.

FIG. 3 is an enlarged top plan view of the shank of FIG. 2.

FIG. 4 is an enlarged bottom plan view of the shank of FIG. 2.

FIG. 5 is an enlarged and partial front elevational view of the shank of FIG. 1.

FIG. 6 is a partial front elevational view of the shank of FIG. 5 with portions broken away to show the detail thereof.

FIG. 7 is a reduced perspective view of the receiver of FIG. 1.

FIG. 8 is a front elevational view of the receiver of FIG. 7 with portions broken away to show the detail thereof.

FIG. 9 is a top plan view of the receiver of FIG. 7.

FIG. 10 is a bottom plan view of the receiver of FIG. 7.

FIG. 11 is an enlarged perspective view of the retainer of FIG. 1.

FIG. 12 is a reduced top plan view of the retainer of FIG. 11.

FIG. 13 is a reduced bottom plan view of the retainer of FIG. 11.

FIG. 14 is an enlarged and partial front elevational view of the retainer of FIG. 11 with portions broken away to show the detail thereof.

FIG. 14 is an enlarged cross-sectional view taken along the line 14-14 of FIG. 12.

FIG. 15 is a perspective view of the insert of FIG. 1.

FIG. 16 is an enlarged front elevational view of the insert of FIG. 15 with portions broken away to show the detail thereof.

FIG. 17 is an enlarged top plan view of the insert of FIG. 15.

FIG. 18 is an enlarged bottom plan view of the insert of FIG. 15.

FIG. 19 is a reduced front elevational view of the receiver, retainer and insert of FIG. 1 with portions of the receiver broken away to show the detail thereof, the retainer being shown downloaded into the receiver and the insert shown in a loading position with respect to the receiver, the insert body pressing the receiver arms outwardly during loading.

FIG. 20 is an enlarged and partial front elevational view similar to FIG. 19 and further showing a subsequent stage of assembly wherein the insert body clears the arms of the receiver during down loading into the receiver.

FIG. 21 is a front elevational view of the insert retainer and receiver with portions broken away, similar to what is shown in FIG. 20 and further showing the insert just prior to rotation of the insert with respect to the receiver.

FIG. 22 is a perspective view of the insert, retainer and receiver assembly with portions of the receiver broken away, similar to what is shown in FIG. 21 and further showing the insert after being rotated within the receiver and also showing the receiver being crimped against the insert to prohibit further rotation of the insert with respect to the receiver.

FIG. 23 is an enlarged and partial front elevational view with portions broken away of the assembly as shown in FIG. 22, and further showing a first stage of assembly with the shank of FIG. 1, also shown in partial front elevation, a hemisphere of the shank head and a vertebra portion are both shown in phantom.

FIG. 24 is a partial front elevational view with portions broken away, similar to FIG. 23 and further showing the shank pressing the retainer up against the insert.

FIG. 25 is a partial front elevational view with portions broken away, similar to FIG. 24, and further showing the shank in a stage of assembly with the retainer, the retainer being in a fully expanded state about a mid-portion of the shank head.

FIG. 26 is a partial front elevational view with portions broken away, similar to FIG. 25, the spherical shank upper portion or head shown fully captured by the retainer.

FIG. 27 is a partial front elevational view with portions broken away, similar to FIG. 26 and further showing the assembly during a pull up or deployment step wherein the receiver is pulled away from the shank, pressing the retainer into a seated relationship with the receiver and also causing the insert to move downwardly in the receiver.

FIG. 28 is a partial front elevational view with portions broken away, similar to FIG. 27 and further showing full deployment of the insert downwardly into the receiver and the shank seated on the retainer that in turn is seated on a lower planar surface of the receiver.

FIG. 29 is a reduced and partial front elevational view with portions broken away, similar to FIG. 28, further showing friction fit non-floppy pivotal movement of the shank with respect to the retainer and the receiver.

FIG. 30 is an enlarged and partial front elevational view with portions broken away, similar to FIG. 29 further shown with a 5.5 mm rod and a closure having a break-off head.

FIG. 31 is a reduced perspective view of the closure of FIG. 30.

FIG. 32 is a top plan view of the closure of FIG. 31.

FIG. 33 is a partial front elevational view with portions broken away, similar to FIG. 30 further showing the closure (with break-off head removed) in locked engagement with the rod.

FIG. 33A is an enlarged and partial front elevational view with portions broken away of the assembly shown in FIG. 33.

FIG. 34 is a partial front elevational view with portions broken away, similar to FIG. 33 but showing the closure (with break-off head removed) in locked engagement with a 6 mm rod in lieu of the 5.5 mm rod.

FIG. 35 is a reduced front elevational view of the assembly of FIG. 34 with the shank shown pivoted to a twenty-six degree angle with respect to the receiver.

FIG. 36 is an enlarged perspective view of an alternative insert having flat panels for use in lieu of the insert shown in FIG. 1.

FIG. 37 is a front elevational view of a partially assembled alternative receiver and insert being shown with a retainer of FIG. 1, the receiver having portions broken away to show the detail thereof.

FIG. 38 is an enlarged perspective view of the insert of FIG. 37.

FIG. 39 is a reduced top plan view of the alternative insert of FIG. 38.

FIG. 40 is a reduced bottom plan view of the alternative insert of FIG. 38.

FIG. 41 is an enlarged front elevational view of the insert of FIG. 38 with portions broken away to show the detail thereof.

FIG. 42 is an enlarged side elevational view of the insert of FIG. 38 with portions broken away to show the detail thereof.

FIG. 43 is an enlarged perspective view of the assembly of FIG. 37 shown pre-assembled and ready for shipping.

FIG. 44 is an enlarged perspective view of the assembly of FIG. 43 with portions broken away to show the detail thereof.

FIG. 45 is an enlarged side elevational view of the assembly of FIG. 43 with portions broken away to show the detail thereof.

FIG. 46 is a reduced and partial front elevational view with portions broken away of the assembly of FIG. 43, and further showing a stage of assembly with the shank of FIG. 1, the shank pressing the retainer up against the insert.

FIG. 47 is a partial front elevational view with portions broken away, similar to FIG. 46, and further showing the shank in a stage of assembly with the retainer, the retainer being in a fully expanded state about a mid-portion of the shank head.

FIG. 48 is a partial front elevational view with portions broken away, similar to FIG. 47, the spherical shank upper portion or head shown fully captured by the retainer.

FIG. 49 is a partial front elevational view with portions broken away, similar to FIG. 48 and further showing the assembly during a pull up or deployment step wherein the receiver is pulled away from the shank, pressing the retainer into a seated position in the receiver.

FIG. 50 is a partial side elevational view with portions broken away of the assembly as shown in FIG. 49 showing the interference fit relationship between the insert and the receiver.

FIG. 51 is a partial side elevational view with portions broken away, similar to FIG. 50 and also showing a subsequent step of pressing the insert further downwardly into the receiver, resulting in a frictional engagement between the insert and the shank wherein the shank is still movable with respect to the insert in a non-floppy manner.

FIG. 52 is a partial front elevational view with portions broken away of the assembly of FIG. 51, further showing the shank being pivoted with respect to the retainer and the receiver.

FIG. 53 is a reduced and partial front elevational view with portions broken away, similar to FIG. 52 and further shown with a 5.5 mm rod and a closure having a break-off head.

FIG. 54 is an enlarged partial perspective view with the rod shown in phantom of the assembly of FIG. 53, further showing the closure (with break-off head removed) in locked engagement with the rod.

FIG. 55 is a partial perspective view, similar to FIG. 54 but showing the closure loosened allowing for manipulation and sliding movement of the rod with respect to the receiver while maintaining the shank in a locked pivotal position with respect to the receiver.

FIG. 56 is a partial perspective view, similar to FIG. 55 further showing the insert after being pulled slightly upwardly, re-mobilizing the assembly to allow for non-floppy pivotal movement of the shank with respect to the receiver.

FIG. 57 is a perspective view of an alternative uni-planar shank for use with the receiver and retainer of FIG. 37.

FIG. 58 is a perspective view of an alternative uni-planar insert for use with the shank of FIG. 57.

FIG. 59 is an enlarged and partial perspective view with portions broken away of the shank of FIG. 57, the insert of FIG. 58 and the retainer and receiver of FIG. 37.

FIG. 60 is an enlarged and partial front elevational view with portions broken away of the assembly of FIG. 59, further shown with a 6 mm rod and the closure of FIG. 53 (with break-off head removed), the assembly being in a locked position.

FIG. 61 is an enlarged and partial side elevational view with portions broken away of the assembly of FIG. 60.

FIG. 62 is a reduced perspective view of the assembly of FIG. 60 further shown with the shank pivoted at an angle with respect to the receiver.

FIG. 63 is a perspective view of an alternative retainer for use with the assembly of FIG. 59 in lieu of the retainer shown in FIG. 59.

FIG. 64 is a reduced front elevational view with portions broken away of the alternative retainer of FIG. 63 shown assembled with the receiver, shank, insert and closure of FIG. 60 and further shown in a locked position with a 5.5 mm rod, a direction of angulation of the shank being in the same plane as the rod.

FIG. 65 is an enlarged and partial perspective view of the insert, retainer and shank of FIG. 64 shown with the receiver, closure and rod removed.

FIG. 66 is an exploded front elevational view of an alternative polyaxial bone screw assembly of an embodiment of the invention including a receiver, an open retainer and an insert, shown with portions broken away to show the detail thereof.

FIG. 67 is a reduced perspective view of the assembly of FIG. 66 with portions broken away to show the detail thereof and showing top loading of the insert into the receiver.

FIG. 68 is a perspective view with portions broken away of the assembly of FIG. 67 shown in a later stage of assembly.

FIG. 69 is a front elevational view with portions broken away, similar to FIG. 68 and further showing the insert fully assembled with the receiver and a shank, shown in partial front elevation being uploaded into the assembly.

FIG. 70 is a partial front elevational view with portions broken away, similar to FIG. 69 showing the shank in a subsequent stage of assembly with the insert.

FIG. 71 is a partial front elevational view with portions broken away, similar to FIG. 70, showing the insert in a subsequent stage of assembly with the receiver and showing the shank being held in friction fit with the insert in a pivoted relation with the receiver.

FIG. 72 is a partial side elevational view with portions broken away of the assembly of FIG. 71 further shown with a rod and a closure, the closure capturing the rod against the insert and the insert pressing the shank into a locked, fixed position within the receiver, the shank shown at an angle of pivot with respect to the receiver of about twenty-five degrees along a run of the rod (which could be directed cephalic or caudal).

FIG. 72A is an enlarged and partial front elevational view of the closure of FIG. 72 with portions broken away to show the detail thereof.

FIG. 73 is a partial perspective view with portions broken away of the assembly of FIG. 71 further shown with a rod and a closure, the closure capturing the rod against the insert and the insert pressing the shank into a locked, fixed position within the receiver (an angle of articulation of the shank with respect to the receiver being shown at about twenty-five degrees medial).

FIG. 74 is a partial perspective view of an alternative bone screw shank for use with bone screw assembly embodiments of the invention.

FIG. 75 is an enlarged and partial front elevational view of the bone screw shank of FIG. 74 with portions broken away to show the detail thereof.

FIG. 76 is a reduced and partial perspective view of the bone screw assembly of FIG. 71 further shown in exploded perspective view with a rigid sleeve, closure and spacer.

FIG. 77 is a partial front elevational view with portions broken away of the bone screw assembly, rigid sleeve, closure and spacer of FIG. 76 and shown assembled with a tensioned cord in phantom.

FIG. 78 is an exploded front elevational view of another alternative polyaxial bone screw assembly of an embodiment of the invention including a receiver, an open retainer and an insert, shown with portions broken away to show the detail thereof.

FIG. 79 is a reduced perspective view of the receiver of FIG. 78.

FIG. 80 is a side elevational view of the receiver of FIG. 79.

FIG. 81 is a reduced and partial front elevational view of the assembly of FIG. 78 with portions broken away to show the detail thereof and is further shown with a shank, also shown in partial front elevation.

FIG. 82 is an enlarged and partial front elevational view with portions broken away of the assembly of FIG. 81 further shown with a rod and a closure.

FIG. 83 is a partial perspective view of the assembly of FIG. 81 further shown with a rigid sleeve a closure, a spacer and a tensioned cord shown in phantom.

FIG. 84 is an exploded front elevational view of another alternative polyaxial bone screw assembly of an embodiment of the invention including a receiver, an open retainer and an insert, shown with portions broken away to show the detail thereof.

FIG. 85 is an exploded reduced perspective view of the assembly shown in FIG. 84.

FIG. 86 is a reduced front elevational view of the assembly of FIG. 86.

FIG. 87 is a front elevational view of the assembly of FIG. 84 with portions broken away further shown assembled with a bone screw shank in partial front elevation and a rod and a closure, also shown in front elevation.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. It is also noted that any reference to the words top, bottom, up and down, and the like, in this application refers to the alignment shown in the various drawings, as well as the normal connotations applied to such devices, and is not intended to restrict positioning of the bone attachment structures in actual use.

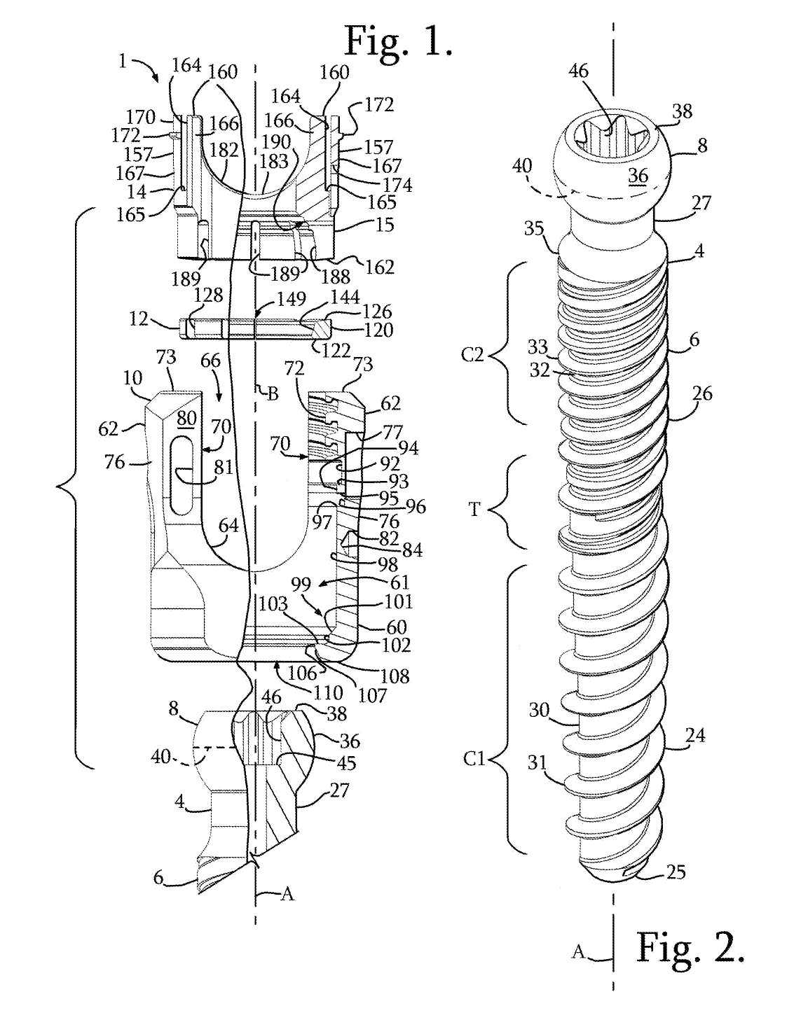

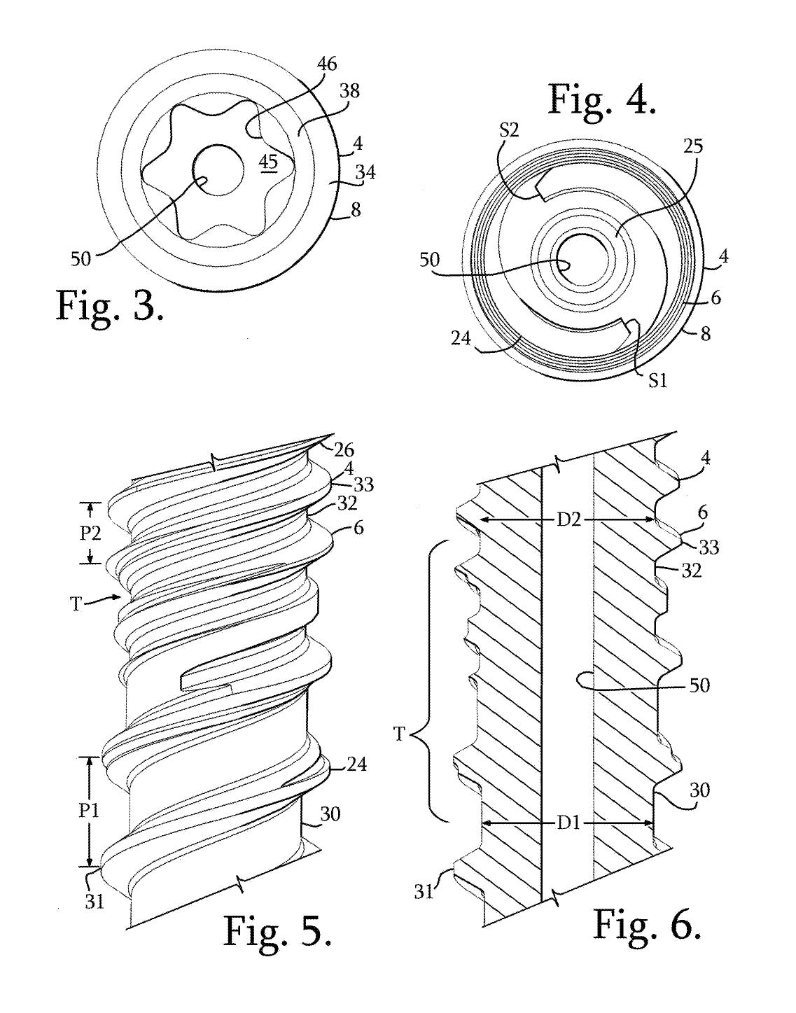

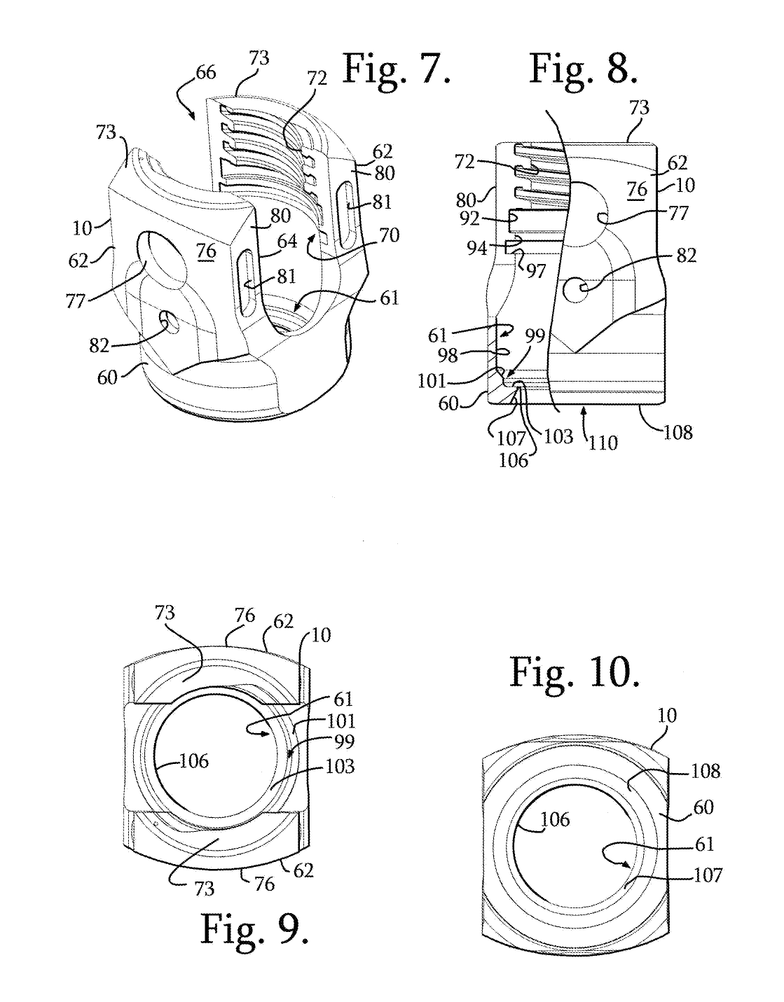

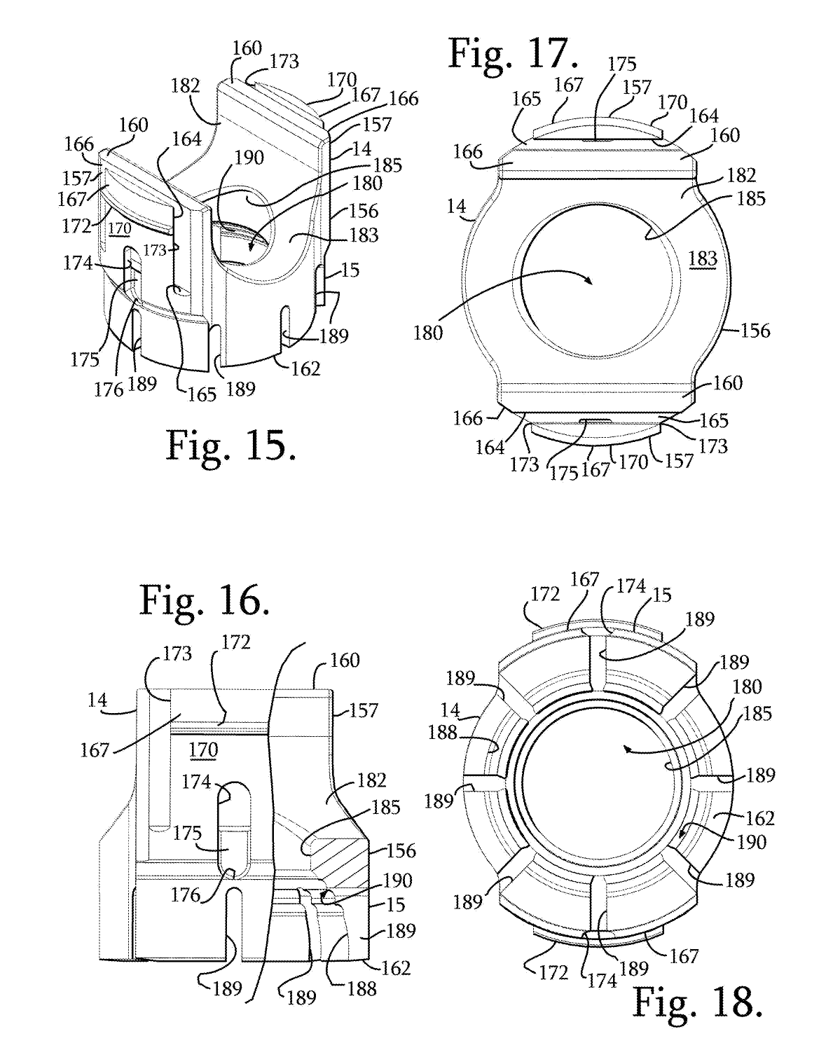

With reference to FIGS. 1-35, the reference number 1 generally represents a polyaxial bone screw apparatus or assembly according to the present invention. The assembly 1 includes a shank 4 having a central axis A, that further includes a body 6 integral with an upwardly extending upper portion or head 8; a receiver 10 having a central axis B; an open retainer 12, and a crown-like compression or pressure insert 14 having an integral lower friction fit portion 15 in the form of a slotted collet. Once assembled with the receiver 10, both the retainer 12 and the insert 14 are substantially coaxial with the receiver 10 with respect to the axis B. The receiver 10, retainer 12 and compression insert 14 are initially assembled and may be further assembled with the shank 4 either prior or subsequent to implantation of the shank body 6 into a vertebra 17, as will be described in greater detail below. FIGS. 30-35 further show a closure structure 18 for capturing a longitudinal connecting member, for example, a rod 21 or 21′ which in turn engages the compression insert 14 that presses against the shank head 8 into fixed frictional contact with the retainer 12, so as to capture and fix the longitudinal connecting member 21 within the receiver 10 and thus fix the member 21 relative to a vertebra 17. The receiver 10 and the shank 4 cooperate in such a manner that the receiver 10 and the shank 4 can be secured at any of a plurality of angles, articulations or rotational alignments relative to one another and within a selected range of angles both from side to side and from front to rear, to enable flexible or articulated engagement of the receiver 10 with the shank 4 until both are locked or fixed relative to each other near the end of an implantation procedure. The illustrated rod 21 has a 5.5 millimeter diameter while the illustrated rod 21′ has a 6.0 millimeter diameter. Both the rods 21 and 21′ are hard, stiff, non-elastic and cylindrical, having a respective outer cylindrical surface 22 and 22′. In some embodiments, the rod may be elastic, deformable and/or of different materials and cross-sectional geometries. It is foreseen that in other embodiments (not shown) the closure top could deform the rod and/or press directly on the insert 14.

The shank 4, best illustrated in FIGS. 1-6, is elongate, with the shank body 6 having a first helically wound dual thread bone implantable thread form 24 with first and second starts S1 and S2 near a bottom or distal end 25 thereof and a second helically wound bone implantable thread 26 with three starts located at an upper or proximal end of the shank 6 near a neck 27 that connects the shank body 6 with the shank upper portion or head 8. Between the thread form 24 and the thread form 26 is a transition area, generally T best shown in FIGS. 5 and 6 wherein the thread forms 24 and 26 connect and morph together. As best shown in FIG. 2, the thread form 24 is located at a greater distance from the shank head 8 and has a length, generally C1 sized for anchoring in cancellous bone. The thread form 26, located near the neck 27 has a length, generally C2 located and configured for engagement in cortical bone. The transition length, generally T spans between C1 and C2. With further reference to FIGS. 5 and 6, the dual thread form 24 has a root surface 30 and a crest surface 31 and the triple thread form 26 has a root surface 32 and a crest surface 33. A virtual cylinder defined by the root surfaces 30 has a minor diameter D1 and a virtual cylinder defined by the root surfaces 32 has a minor diameter D2. The minor root diameters D1 and D2 are substantially equal along the transition T length of the shank as well as the cancellous length C1 and the cortical length C2. During manufacture of the shank 6 care is taken to ensure that along the transition length T where the thread form 24 morphs into the thread form 26, the minor diameter remains substantially constant. Although, as illustrated in FIG. 6, crest portions 31 and 33 may be reduced or removed in places along the transition length T where the thread forms 24 and 26 intersect, a major diameter of the shank at the transition length T, which can be defined as a diameter of a virtual cylinder formed by the thread form crests, is never greater than a major diameter of the thread form 24 or a major diameter of the thread form 26. The transition from a dual lead or start form 24 to a triple lead or start form 26 results in the shank 6 that has a thread form for gripping cancellous bone with a pitch P1 and another thread form for gripping cortical bone with a pitch P2 wherein P1 is greater than P2, but such difference in pitch is small in degree and thus provides for a relatively smooth transition between thread forms during insertion of the screw into bone. The smaller pitch P2 along the screw length C2 allows for an increased surface area without slowing down an advance rate of the screw into bone, resulting in a desirable near constant advancement speed without push or pull. During manufacture of the screw body 6, rather than interweaving or interleaving thread forms as is known in the prior art, two distinct thread patterns are machined and as shown in FIG. 6, the small transition area or length T is provided wherein the thread form 24 relatively smoothly and gradually changes into the thread form 26. Thus, it is not necessary to have integral multiples of shank threads (e.g., lower two start form transition to an upper four start form) required by an inter-weaving or -leafing process and the associated less desirable greater difference in pitch between lower and upper sections of the shank body. For example, it is foreseen that another desirable thread form transition according to the invention is a three start helically wound lower thread form section for gripping cancellous bone that transitions into a five start thread form for gripping cortical bone.

With further reference to FIGS. 2 to 4, during use, the body 6 utilizing the threads 24 and 26 for gripping and advancement is implanted into the vertebra 17 leading with the tip 25 and driven down into the vertebra with an installation or driving tool (not shown), so as to be implanted in the vertebra to a location at or near an end 35 of the thread form 26 located near the neck 27. As stated above, the shank 4 has an elongate axis of rotation generally identified by the reference letter A.

The neck 27 extends axially upwardly from the shank body 6. The neck 27 may be of the same or is typically of a slightly reduced radius as compared to the adjacent upper end or top 35 of the body 6 where the thread form 26 terminates. Further extending axially and outwardly from the neck 26 is the shank upper portion or head 8 that provides a connective or capture apparatus disposed at a distance from the upper end 35 and thus at a distance from the vertebra 17 when the body 6 is implanted in such vertebra.

The shank upper portion 8 is configured for a pivotable connection between the shank 4 and the retainer 12 and receiver 10 prior to fixing of the shank 4 in a desired position with respect to the receiver 10. The shank upper portion 8 has an outer, convex and substantially spherical surface 36 that extends outwardly and upwardly from the neck 26 to a top surface or rim 38. In some embodiments, a frusto-conical surface is located between the spherical surface 36 and the rim 38 to provide for greater angulation of the shank with respect to the receiver, providing additional clearance during pivoting of the shank with respect to the receiver 10 and the insert 14. The spherical surface 36 has an outer radius configured for temporary frictional, non-floppy, sliding cooperation with the lower collet portion 15 of the insert 14 as well as ultimate frictional engagement with the insert 14 and the retainer 12 at a lower inner edge or surface thereof. In FIG. 2 and some of the other figures, a dotted line 40 designates a hemisphere of the spherical surface 36. The spherical surface 36 shown in the present embodiment is substantially smooth, but in some embodiments may include a roughening or other surface treatment and is sized and shaped for cooperation and ultimate frictional engagement with the compression insert 14 as well as ultimate frictional engagement with an inner surface portion of the retainer 12. The shank spherical surface 36 is locked into place exclusively by the insert 14 and the retainer 12 surface portion and not by inner surfaces defining the receiver 10 cavity.

A substantially planar counter sunk annular seating surface or base 45 partially defines a portion of an internal drive feature or imprint 46. The illustrated internal drive feature 46 is an aperture formed in the top 38 and has a star shape designed to receive a tool (not shown) of an Allen wrench type, into the aperture for rotating and driving the bone screw shank 4 into the vertebra 17. It is foreseen that such an internal tool engagement structure may take a variety of tool-engaging forms and may include one or more apertures of various shapes, such as a pair of spaced apart apertures or a hex shape or a multi-lobular aperture, for example. The seat or base surface 45 of the drive feature 46 is disposed substantially perpendicular to the axis A with the drive feature 46 otherwise being coaxial with the axis A. In some embodiments, the drive seat 45 may have beveled or stepped surfaces for further enhancing gripping with the driving tool. In operation, a driving tool is received in the internal drive feature 46, being seated at the base 45 and engaging the faces of the drive feature 46 for both driving and rotating the shank body 6 into the vertebra 17, either before or after the shank 4 is connected to the receiver 10 via the retainer 12, the driving tool extending into the receiver 10 and the insert 14 when a pre-assembled shank 4, retainer 12, insert 14 and receiver 10 bone screw assembly is driven into the vertebra 17.

The shank 4 shown in the drawings is cannulated, having a small central bore 50 extending an entire length of the shank 4 along the axis A. The bore 50 is defined by an inner cylindrical wall of the shank 4 and has a circular opening at the shank tip 25 and an upper circular opening communicating with the external drive 46 at the driving seat 45. The bore 50 is coaxial with the threaded body 6 and the upper portion or head 8. The bore 50 provides a passage through the shank 4 interior for a length of wire (not shown) inserted into the vertebra 17 prior to the insertion of the shank body 6, the wire providing a guide for insertion of the shank body 6 into the vertebra 17. It is foreseen that the shank could be solid and made of different materials, including metal and non-metals. As will be discussed in greater detail below, preferably, the shank is made from a material that is not as hard as a material or materials used to make the retainer 12 and the insert 14.

To provide a biologically active interface with the bone, the threaded shank body 6 may be coated, perforated, made porous or otherwise treated. The treatment may include, but is not limited to a plasma spray coating or other type of coating of a metal or, for example, a calcium phosphate; or a roughening, perforation or indentation in the shank surface, such as by sputtering, sand blasting or acid etching, that allows for bony ingrowth or ongrowth. Certain metal coatings act as a scaffold for bone ingrowth. Bio-ceramic calcium phosphate coatings include, but are not limited to: alpha-tri-calcium phosphate and beta-tri-calcium phosphate (Ca₃(PO₄)₂, tetra-calcium phosphate (Ca₄P₂O₉), amorphous calcium phosphate and hydroxyapatite (Ca₁₀(PO₄)₆(OH)₂). Coating with hydroxyapatite, for example, is desirable as hydroxyapatite is chemically similar to bone with respect to mineral content and has been identified as being bioactive and thus not only supportive of bone ingrowth, but actively taking part in bone bonding.

With particular reference to FIGS. 1 and 7-10, the receiver 10 has a generally U-shaped appearance with partially discontinuous cylindrical inner and outer profiles as well as planar and other curved surfaces. The receiver 10 has an axis of rotation B that is shown in FIG. 1 as being aligned with and the same as the axis of rotation A of the shank 4, such orientation being desirable, but not required during assembly of the receiver 10 with the shank 4. After the receiver 10 is pivotally attached to the shank 4, either before or after the shank 4 is implanted in a vertebra 17, the axis B is typically disposed at an angle with respect to the axis A.

The receiver 10 includes a base 60 forming an inner cavity, generally 61. Two opposed arms 62 extend upwardly from the base 60 and form a U-shaped channel 64 having an opening 66. Other features of the receiver 10 include, but are not limited to inner receiver arms surfaces, generally 70 that include a guide and advancement structure 72 located near arm top surfaces 73. In the illustrated embodiment, the guide and advancement structure 72 is a partial helically wound interlocking flangeform configured to mate under rotation with a similar structure on the closure structure 18. However, it is foreseen that for certain embodiments of the invention, the guide and advancement structure 72 could alternatively be a square-shaped thread, a buttress thread, a reverse angle thread or other thread-like or non-thread-like helically wound discontinuous advancement structures, for operably guiding under rotation and advancing the closure structure 18 downward between the arms 62, as well as eventual torquing when the closure structure 18 abuts against the rod 21 or other longitudinal connecting member. It is foreseen that the arms 62 could have break-off extensions.

Each arm 62 has an outer surface 76 with one or more tool receiving grooves, recesses or apertures. In the illustrated embodiment a first tool receiving recess 77 is cylindrical in form and centrally located between arm side surfaces 80 and near, but spaced from the top surface 73. Each side surface 80 also has an oblong recess 81 (total of four recesses) that may be used to receive portions of manipulating tools. The recesses 77 and 81 do not extend all the way through the respective arm portions. Another cylindrical recess 82 is formed centrally in each arm below each recess 77. Each recess 82 is partially defined by a thin wall that provides a crimping portion or wall 84. The total of two crimping portions or walls 84 are sized and shaped for pressing or crimping some or all of the wall material into walls or grooves of the insert 14 to prohibit rotation and misalignment of the insert 14 with respect to the receiver 10 as will be described in greater detail below. In other embodiments of the invention, other surfaces or grooves may be inwardly crimped. The receiver 10 is a one-piece or integral structure and is devoid of any spring tabs or collet-like structures. Preferably the insert and/or receiver are configured with structure for blocking rotation of the insert with respect to the receiver, such as the crimp walls 84, but allowing some up and down movement of the insert with respect to the receiver during the assembly and implant procedure.

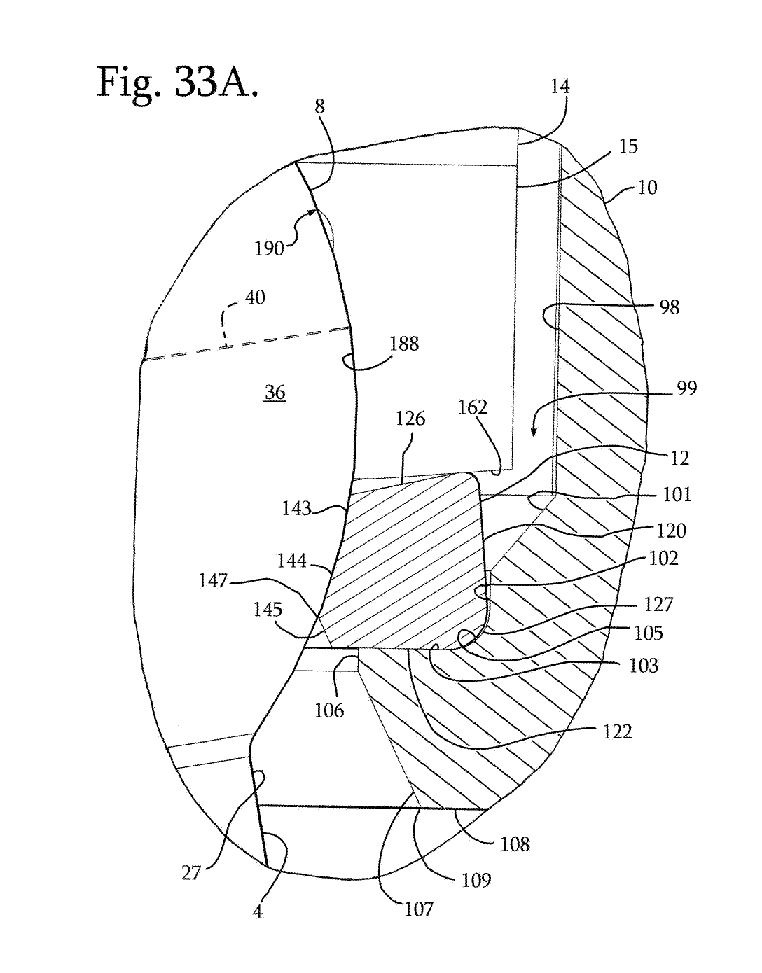

With particular reference to FIGS. 1 and 8 and also with reference to FIG. 33A, returning to the interior surface 70 of the receiver arms 62, located below the guide and advancement structure 72 is a discontinuous cylindrical surface 92 partially defining a run-out feature for the guide and advancement structure 72. The cylindrical surface 92 is sized and shaped to receive an upper portion of the insert 14. Therefore, the surface 92 has a diameter greater than a greater diameter of the guide and advancement structure 72. The receiver arms may further include sloped, stepped or chamfered surfaces above and below the surface 92. Directly below the surface 92 is a lip or ledge surface 93 that extends inwardly toward the axis B and functions as a stop for the insert 14. A discontinuous cylindrical surface 94 extends downwardly from the ledge surface 93, the surface 94 being parallel to the axis B and having a diameter smaller than the diameter of the cylindrical surface 92. Adjacent the surface 94 is a discontinuous annular surface 95 that is perpendicular to the axis B and extends outwardly to near the recess 77. Adjacent and below the surface 95 is another discontinuous cylindrical surface 96, the surface 96 being parallel to the axis B and having a diameter larger than the diameter of the cylindrical surface 92. Adjacent and below the surface 96 is a discontinuous annular surface or ledge 97 that is perpendicular to the axis B. The ledge surface 97 extends from the cylindrical surface 96 inwardly to a cylindrical surface 98 that defines lower portions of the receiver inner arms 70 as well as a portion of the base cavity 61. The cylindrical surface 98 is also parallel to the axis B and has a diameter that is smaller than the diameter of the surface 96. In the illustrated embodiment the diameter of the surface 98 and the diameter of the surface 94 are the same. The diameter of the surface 98 is sized and shaped to allow for expansion of the retainer 12 about the shank upper portion 8 within the receiver cavity 61. The surface 98 terminates at a lower stepped or tiered retainer seating and expansion locking portion, generally 99, that includes a substantially frusto-conical surface 101 adjacent the surface 98, a cylindrical surface 102, a bottom annular and planar seating or loading surface 103, a rounded or radiused corner portion 105 connecting the surface 102 with the surface 103, a lower flared or tapered surface 107 opening to a bottom exterior surface 108 at a bottom opening, generally 110 of the receiver. The seating surface 103 terminates at a narrow cylindrical surface 106 that connects the seating surface 103 with the tapered surface 107. The surface 106 is substantially parallel to the axis B and has a diameter smaller than a diameter of the surface 102, the surface 102 also being substantially parallel to the axis B. The surface 106 diameter is also smaller than a diameter of a lower opening edge 109 formed at the intersection of the surface 107 and the surface 108. It is noted that additional curved or radiused surfaces may be included in the seating portion 99 to provide for a graduated transition from the expansion chamber defined by the surface 98 to the planar retainer seat 103.

With particular reference to FIGS. 1 and 11-14, the lower open or split friction fit retainer 12, that operates to capture the shank upper portion 8 within the receiver 10 is shown. The retainer 12 has a central axis that is operationally the same as the axis B associated with the receiver 10 when the shank upper portion 8 and the retainer 12 are installed within the receiver 10. The retainer 12 is essentially an open ring having an outer cylindrical surface 120, a bottom substantially planar and annular surface 122, and a top surface 126 that slopes downwardly and inwardly from the outer surface 120 toward the axis B in a curved or slightly radiused or frusto-conical fashion toward the central axis B. A lower radiused corner surface portion 127 connects the outer surface 120 with the bottom surface 122.

Outer spaced grooves or notches 128 are formed in the cylindrical surface 120 and run through the top surface 126 and the bottom surface 122. The illustrated ring 12 includes eight equally spaced notches 128. Fewer or greater numbers of notches are foreseen. The illustrated notches are partially cylindrical and extend radially inwardly a distance of about halfway through a radial thickness of the ring. However, notches formed more or less deeply into one or more surfaces of the ring 12 are foreseen.

The number and depth of the notches may vary depending upon the hardness of the material used to make the ring 12. When the retainer is made from a more resilient material, such as stainless steel or titanium, the ring may not require any notches or may require one or a pair of spaced notches, for example. When the retainer is made from a less resilient material that is harder than the material or materials used for the shank 4 and the receiver 10, such as cobalt chrome, a plurality of notches is desired to provide a desired resiliency. Cobalt chrome (Co—Cr) is a metal alloy of cobalt and chromium having a very high specific strength and, in some embodiments may further include molybdenum. Cobalt-chromium alloys are desirable as they are strong, hard, bio-compatible and corrosion resistant.

The retainer 12 has a central channel or hollow through bore, generally 141, that passes entirely through the retainer 12 from the top surface 126 to the bottom surface 122 of the retainer body. Surfaces that define the channel or bore 141 include a discontinuous inner upper surface 143 located adjacent the top surface 126 that is radiused or may be frusto-conical. The surface 143 is also adjacent a lower radiused surface 144 that terminates at or near a flared or frusto-conical surface 145. In the illustrated embodiment a narrow cylindrical surface 147 connects the surface 144 with the surface 145. In the illustrated embodiment, the surface 144 has a radius that is substantially the same as a radius of the shank upper portion 8 surface 36, while the surface 143 has a slightly larger radius than the radius of the surface 144. In some embodiments of the invention, the surfaces 143 and 144 may be replaced by a single radiused surface having a radius substantially similar to the radius of the shank surface 36. In other embodiments an inner edge may be defined by radiused or frusto-conical surfaces to create an edge lock between the retainer and the shank head. As is shown in FIG. 33A, and as will be described in greater detail below, when the retainer 12 is made from a titanium alloy, for example, the notched retainer 12 may resiliently move in response to downward pressure from the spherical shank head 8 during final locking so that when the surfaces 143 and 144, or portions thereof, frictionally engage the surface 36 of the shank head 8, and an upper portion of the outer surface 120 may move away from the receiver surface 102, the resilient and flexible retainer “folding in” slightly in response to the locking force due to a decreased strength of the retainer that includes the plurality of notches 128. However, the receiver annular and substantially planar surface 103 adequately supports the retainer, guarding against undesirable pull-out of the retainer even if such “folding in” occurs during final locking. It has been found, however, that when the retainer 12 is made from a harder material, such a cobalt-chrome, such “folding in” of the retainer does not occur. The cobalt-chrome retainer surface 120 does not pull away from the surface 102, even when there are notches formed in the surface 120, the surface 120 remaining in engagement with the surface 102 during final locking of the polyaxial mechanism when the insert 14 is pressed downwardly into locked frictional engagement with the shank head 8.

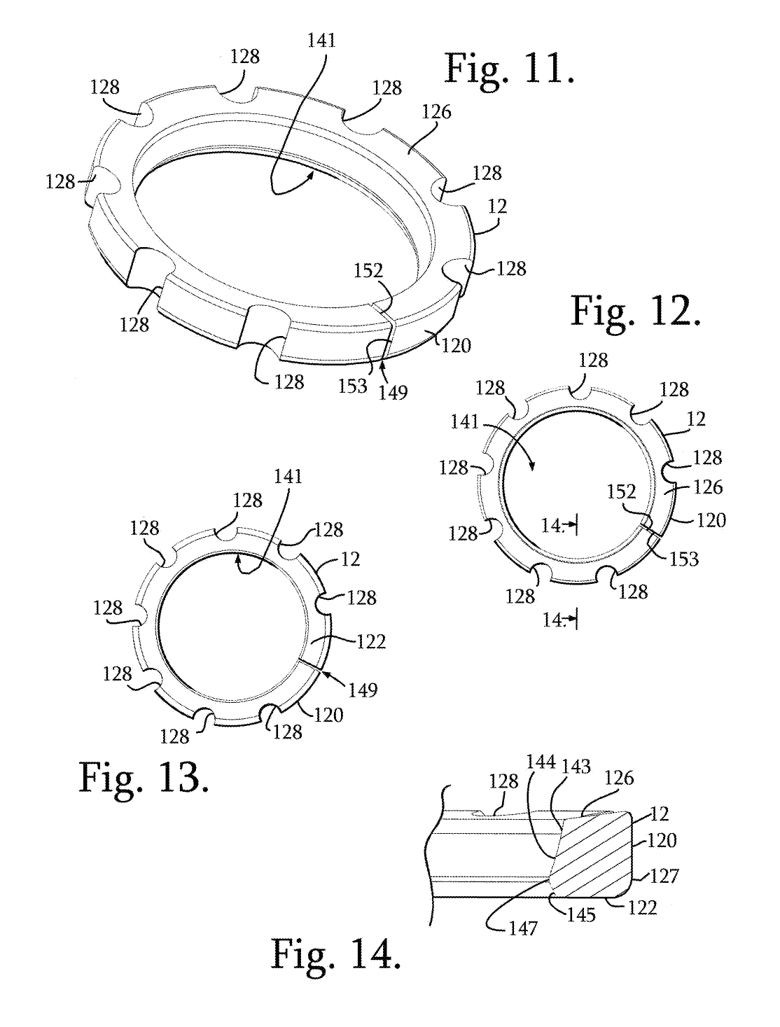

A slit, generally 149 runs through the retainer 14, creating an opening generally perpendicular to the bottom surface 122. The slit 149 is primarily for expansion of the retainer 12 during pop-on or snap-on assembly with the shank head 8. The through slit 149 of the resilient retainer 12 is defined by first and second end surfaces, 152 and 153 disposed in substantially parallel spaced relation to one another when the retainer is in a neutral or nominal state. Both end surfaces 152 and 153 are disposed perpendicular to the bottom surface 122, but in some embodiments may be disposed at an obtuse angle thereto. A width between the surfaces 152 and 153 is narrow to provide stability to the retainer 12 during operation, but wide enough to allow for some compression of the retainer during assembly, if needed. Because the retainer 12 is top loadable in a substantially neutral state and ultimately expands during locking of the polyaxial mechanism, the width of the slit 149 may be much smaller than might be required for a bottom loaded compressible retainer ring. It has been found that once the retainer 12 is expanded about the shank head 8, the retainer 12 may return to a new nominal or neutral orientation in which a gap between the surfaces 152 and 153 is slightly greater than the gap shown in the nominal state of FIG. 11, for example.

With particular reference to FIGS. 1 and 15-18, the compression insert 14 with the integral lower friction fit compressive collet 15 is illustrated that is sized and shaped to be received by and down-loaded into the receiver 10 at the upper opening 66. The compression insert 14 has an operational central axis that is the same as the central axis B of the receiver 10. Features of the friction fit insert 14 include an upper body 156 integral with a pair of upstanding arms 157. The lower body or collet 15 extends downwardly and axially from the upper body 156 and is also substantially cylindrical in outward appearance. Substantially planar arm top surfaces 160 are located opposite bottom surfaces 162 of the collet portion 15. Each of the arms 157 includes a slot 164 cut into the top surface 160 and running downwardly to a slot U-shaped base surface 165 located spaced from the friction fit collet portion 15. The arm slots 164 are parallel to one another and to the axis B. The slots 164 separate each arm 157 into an inner arm portion 166 and an outer portion 167, each outer portion 167 being resilient and pressable toward each inner portion 166. Each outer portion includes a discontinuous outer cylindrical surface 170. Extending from each surface 170 and located near but spaced from the arm top 160 is a radially projecting strip or lip 172 extending to either side 173 of the arm outer portion 167 and running in a plane substantially parallel to each arm top surface 160. Located centrally below each strip 172 and spaced therefrom is an oblong recess 174 oriented generally perpendicular to the arm top surface 160, an upper portion of which extends completely through the respective arm outer portion 167 and thus communicates with the slot 164. A lower portion of the recess 174 is partially defined by a back wall surface 175. The wall 175 extends downwardly and terminates at a U-shaped surface 176 that is adjacent the collet portion 15. The recess 174 and the wall 175 are sized and shaped for receiving a crimped wall portion 84 of the receiver 10 as will be described in greater detail below.

Returning to the inner surfaces of the insert 14, a through bore, generally 180, is disposed primarily within and through the insert 14 and communicates with a generally U-shaped through channel formed by a saddle surface 182 that is substantially defined by the upstanding arms 157. Near the top surfaces 160, the saddle surface 182 is substantially planar. The saddle 182 has a lower seat 183 sized and shaped to closely, snugly engage the 6 mm rod 21′ or other longitudinal connecting member. It is foreseen that an alternative embodiment may be configured to include planar holding surfaces that closely hold a square or rectangular bar as well as hold a cylindrical rod-shaped, cord, or sleeved cord longitudinal connecting member. The saddle 183 and the closure 18 cooperate to fix the smaller 5.5 mm rod 21 against a portion of the saddle 183 as will be described in greater detail below.

The bore, generally 180, is further defined by an inner cylindrical surface 185 that communicates with the seat 183 and a lower concave, radiused inner collet surface 188 that terminates at the bottom surface 162, the illustrated surface 188 having a radius sized and shaped for frictionally engaging the surface 36 of the shank upper portion 8. The inner collet surface 188 is discontinuous, being broken up by eight spaced grooves 189 that run from the bottom surface 162 upwardly toward the insert upper body 156, terminating at a shank gripping surface portion, generally 190. In some embodiments, each of the collet surfaces 188 are planar rather than radiused with a portion of each such planar surfaces pressing against the shank surface 36. The gripping surface 190 spans from the cylindrical surface 185 to the lower radiused surface 188. The gripping surface portion 190 includes more than one and up to a plurality of stepped surfaces or ridges sized and shaped to grip and penetrate into the shank head 8 when the insert 14 is finally locked against the head surface 36. The illustrated gripping portion 190 includes at least three ridges or edges. It is foreseen that the shank gripping surface portion 190 and also the surface 188 may additionally or alternatively include a roughened or textured surface or surface finish, or may be scored, knurled, or the like, for enhancing frictional engagement with the shank upper portion 8.

The compression insert 14 through bore 180 is sized and shaped to receive a driving tool therethrough that engages the shank drive feature 46 when the shank body 6 is driven into bone with the receiver 10 attached. Also, in some locking embodiments of the invention, the bore may receive a manipulation tool used for releasing the insert from a locked position with the receiver, the tool pressing down on the shank and gripping the insert at tool engaging features. Each of the arms 157 and the insert body 156 may include more surface features, such as cut-outs notches, bevels, etc. to provide adequate clearance for inserting the insert 14 into the receiver and cooperating with the retainer 12 during the different assembly steps.

The insert body 156 has an outer diameter slightly greater than a diameter between crests of the guide and advancement structure 72 of the receiver 10. Thus, as will be described below, the insert 14 presses the receiver arms 62 outwardly away from one another during top loading of the compression insert 14 into the receiver opening 66. Thus, a desirable material for the receiver 10 is a more resilient material such as a titanium alloy, while a desirable material for the insert 14 is a harder material, such as cobalt-chrome. Once the arms 157 of the insert 14 are generally located beneath the guide and advancement structure 72, the insert 14 body 156 has cleared the structure 72 and can be rotated into place about the receiver axis B with the radially extending strips 172 entering the receiver groove formed by the cylindrical surface 92.

With reference to FIGS. 30 and 33-35, for example, the illustrated elongate rods or longitudinal connecting members 21 (5.5 mm diameter) and 21′ (6.0 mm diameter), of which only portions have been shown, can be any of a variety of implants utilized in reconstructive spinal surgery, but are typically a cylindrical, elongate structure having an outer substantially smooth, cylindrical surface 22 or 22′ of uniform diameter. The rod 21 or 21′ may be made from a variety of metals, metal alloys, non-metals and deformable and less compressible plastics, including, but not limited to rods made of elastomeric, polyetheretherketone (PEEK) and other types of materials, such as polycarbonate urethanes (PCU) and polyethelenes.

Longitudinal connecting members for use with the assembly 1 may take a variety of shapes, including but not limited to rods or bars of oval, rectangular or other curved or polygonal cross-section. The shape of the insert 14 may be modified so as to closely hold the particular longitudinal connecting member used in the assembly 1. Some embodiments of the assembly 1 may also be used with a tensioned cord, with or without rigid sleeves for holding the cord. Such a cord may be made from a variety of materials, including polyester or other plastic fibers, strands or threads, such as polyethylene-terephthalate. Furthermore, the longitudinal connector may be a component of a longer overall dynamic stabilization connecting member, with cylindrical or bar-shaped portions sized and shaped for being received by the compression insert 14 of the receiver having a U-shaped, rectangular- or other-shaped channel, for closely receiving the longitudinal connecting member. The longitudinal connecting member may be integral or otherwise fixed to a bendable or damping component that is sized and shaped to be located between adjacent pairs of bone screw assemblies 1, for example. A damping component or bumper may be attached to the longitudinal connecting member at one or both sides of the bone screw assembly 1. A rod or bar (or rod or bar component) of a longitudinal connecting member may be made of a variety of materials ranging from deformable plastics to hard metals, depending upon the desired application. Thus, bars and rods may be made of materials including, but not limited to metal and metal alloys including but not limited to stainless steel, titanium, titanium alloys and cobalt chrome; or other suitable materials, including plastic polymers such as polyetheretherketone (PEEK), ultra-high-molecular weight-polyethylene (UHMWP), polyurethanes and composites, including composites containing carbon fiber, natural or synthetic elastomers such as polyisoprene (natural rubber), and synthetic polymers, copolymers, and thermoplastic elastomers, for example, polyurethane elastomers such as polycarbonate-urethane elastomers.

With reference to FIGS. 30-35, for example, the closure structure or top 18 shown with the assembly 1 is rotatably received between the spaced arms 62 of the receiver 10. It is noted that the closure 18 could be a twist-in or slide-in closure structure. The illustrated closure structure 18 has a substantially cylindrical body 191 that includes an outer helically wound flange form guide and advancement structure 192 (dual start) that operably joins with the guide and advancement flange form structure 72 disposed on the arms 62 of the receiver 10. It is noted that other multi-start or single start forms may be used. The particular geometry of the flange form structure utilized in accordance with certain embodiments of the invention may take a variety of forms, including those described in Applicant's U.S. Pat. No. 6,726,689, which is incorporated herein by reference. Although it is foreseen that the closure guide and advancement structure 192 could alternatively be in the for of a buttress thread, a square thread, a reverse angle thread or other thread-like or non-thread-like helically wound advancement structure, for operably guiding under rotation and advancing the closure 18 downward between the arms 62 and having such a nature as to resist splaying of the arms 62 when the closure 18 is advanced into the channel 64. The flange form geometry illustrated herein and as described more fully in Applicant's U.S. Pat. No. 6,726,689 is preferred as the added strength provided by such flange form beneficially cooperates with and counters any reduction in strength caused by the any reduced profile of the receiver 10 that may more advantageously engage longitudinal connecting member components. The illustrated closure structure 18 also includes a break-off head 193 having a hex shape sized and shaped for cooperation with a socket-type tool. The head 193 is designed to break from the body 191 of the closure at a preselected torque, for example, 70 to 140 inch pounds. The closure body 191 includes a top surface 194 and an internal drive 196 formed therein that defines an aperture and is illustrated as a star-shape, such as that sold under the trademark TORX, or may be, for example, a hex drive or other internal drives such as slotted, tri-wing, spanner, two or more apertures of various shapes, and the like. A driving tool (not shown) sized and shaped for engagement with the internal drive 196 may be used for both rotatable disengagement of the closure 18 from the receiver arms 62, and re-engagement, if required. A base or bottom surface 197 of the closure is planar and further includes a central dome or nub 198 for gripping of a rod and is particularly desirable for positioning the 5.5 mm rod 21 as will be described in greater detail below. The illustrated nub 198 extends axially downwardly away from a mound or more shallow radiused portion or projection 199 that extends downwardly from the planar bottom surface 197, The mound 199 forms an annular gradient or rim surrounding the nub 198, the mound 199 having a radius that is greater than a radius of the nub 198. The nub 198 is also desirable for use with deformable rods. In other embodiments, closure tops may include central points and/or spaced outer rims for engagement and penetration into the surface 22 or 22′ of the respective rod 21 or 21′. It is noted that in some embodiments, the closure bottom surface does not include a nub, point, or rim. In some embodiments, the closure may further include a cannulation through bore extending along a central axis thereof, opening at the drive feature and extending through the bottom surfaces thereof. Such a through bore provides a passage through the closure interior for a length of wire (not shown) inserted therein to provide a guide for insertion of the closure top into the receiver arms 62.

The assembly 1 receiver 10, retainer 12 and compression insert 14 are typically assembled at a factory setting that includes tooling for holding and alignment of the component pieces and manipulating the retainer 12 and the insert 14 with respect to the receiver 10. In some circumstances, the shank 4 is also assembled with the receiver 10, the retainer 12 and the compression insert 14 at the factory. In other instances, it is desirable to first implant the shank 4, followed by addition of the pre-assembled receiver, retainer and compression insert at the insertion point. In this way, the surgeon may advantageously and more easily implant and manipulate the shanks 4, distract or compress the vertebrae with the shanks 4 and work around the shank upper portions or heads 8 without the cooperating receivers 10 being in the way. In other instances, it is desirable for the surgical staff to pre-assemble a shank 4 of a desired size and/or variety (e.g., surface treatment of roughening the upper portion 8 and/or hydroxyapatite on the shank 6), with the receiver 10, retainer 12 and compression insert 14. Allowing the surgeon to choose the appropriately sized or treated shank 4 advantageously reduces inventory requirements, thus reducing overall cost and improving logistics and distribution.

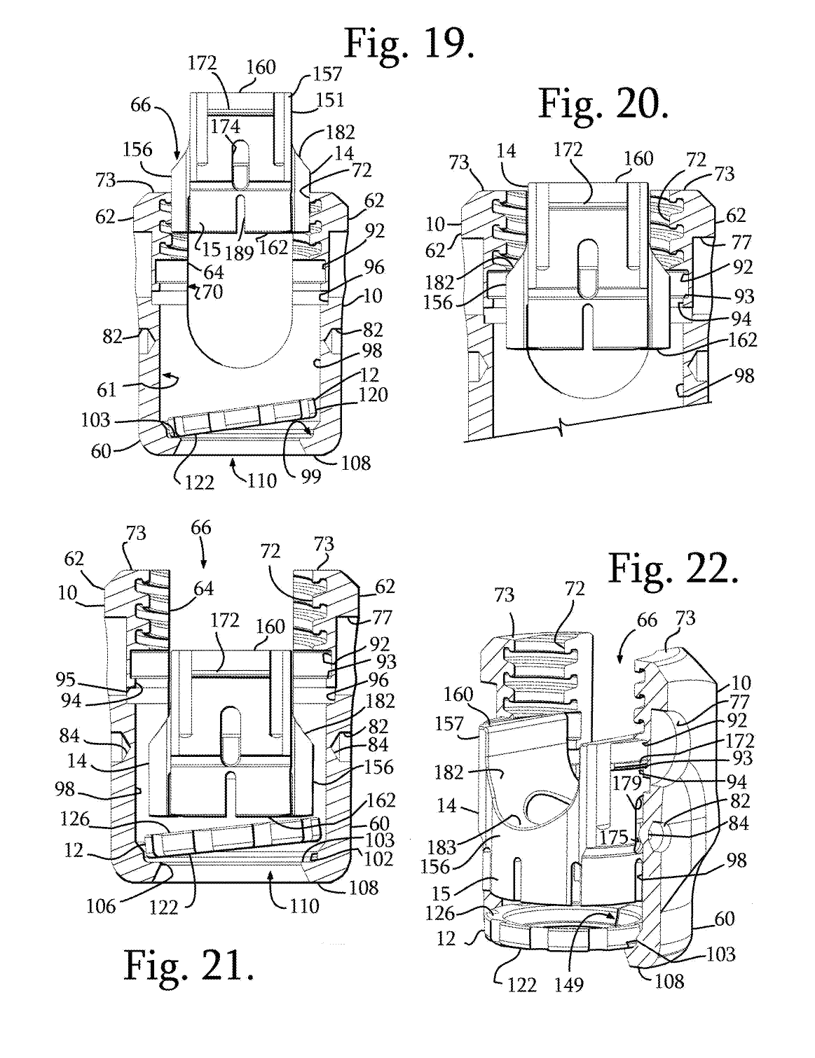

Pre-assembly of the receiver 10, retainer 12 and compression insert 14 is shown in FIGS. 19-22. With particular reference to FIG. 19, first the retainer 12 is inserted into the upper receiver opening 66, leading with the outer surface 120 with the top surface 126 facing one arm 62 and the retainer bottom surface 122 facing the opposing arm 62. The retainer 12 is then lowered in such sideways manner into the channel 64 and partially into the receiver cavity 61, followed by tilting the retainer 12 to a position wherein the central axis of the retainer 12 is generally aligned with the receiver central axis B as shown in FIG. 19, with some or all of the retainer bottom surface 122 resting on the receiver seating surface 103. The retainer 12 is free to rotate with respect to the receiver about the axis B.

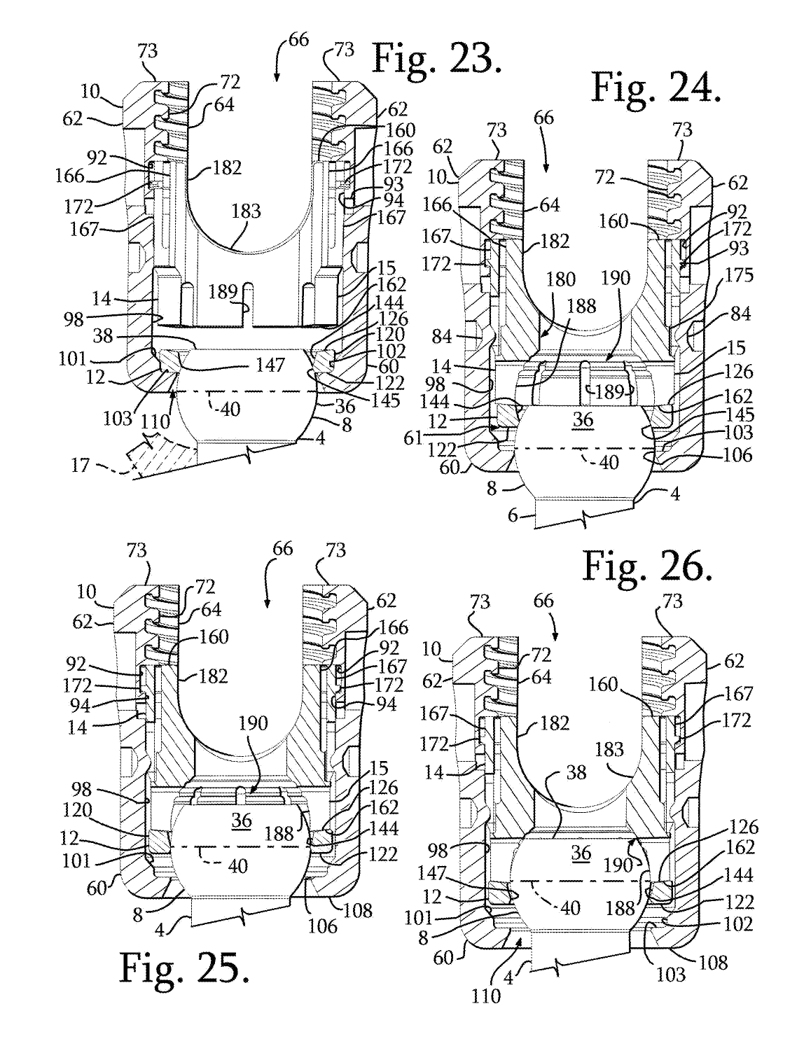

With further reference to FIG. 19 and with reference to FIGS. 20 and 21, the compression insert 14 is then downloaded into the receiver 10 through the upper opening 66 with the bottom surface 162 facing the receiver arm top surfaces 73 and the insert arms 157 located between the opposed receiver arms 62. The insert 14 is then lowered toward the receiver and between the arms 62 with the insert body 156 initially in a tight or press fit arrangement with the receiver 10 at the guide and advancement structures 72 located on the inner surfaces 70 near the top surfaces 73 of the arms 62. Force is used to move the insert body 156 between the guide and advancement structures 72, slightly splaying the arms 62 away from on another. As indicated previously, the receiver 10 is preferably made from a resilient material such as a stainless steel or titanium alloy, to allow for a temporary outward splaying of the arms 62 during initial insertion of the insert 14. Also, as indicated previously, a preferred material for the insert 14 is a cobalt-chrome alloy that is harder than a material of the receiver 10. With reference to FIG. 20, as soon as the body 156 of the insert 14 clears the guide and advancement structures 72 and is situated within the receiver cylindrical surfaces 92, 94, 96 and 98, the resilient receiver arms 62 return to an original orientation and the insert 14 is now captured within the receiver 10, also capturing the retainer 12 within the receiver 10. With reference to FIG. 21, the insert 14 is then lowered to a position wherein the insert 14 arm top surfaces 160 are adjacent to the run-out area below the guide and advancement structure 72 defined in part by the cylindrical surface 92. With reference to FIG. 22, thereafter, the insert 14 is rotated about the receiver axis B until the upper arm surfaces 160 are directly below the guide and advancement structure 72 with the radially projecting strips or lips 172 located adjacent to cylindrical surfaces 92 of the receiver and resting on the annular surfaces 93 as also shown in FIGS. 23-26. In some embodiments, the insert arm outer portions 167 may need to be compressed slightly inwardly during rotation to clear some of the inner surfaces 70 of the receiver arms 62. With reference to FIG. 22, the insert 14 is now captured in a desired shipping position wherein the guide and advancement structures 72 of the receiver 10 prohibit upward movement of the insert 14 and the annular surfaces 93 prohibit downward movement of the insert 14. Also, with further reference to FIG. 22, after the insert 14 is rotated about the axis B to a desired aligned position with respect to the receiver, the insert channel 182 being aligned with the receiver channel 64, the opposed crimping walls 84 now located adjacent the oblong recesses 174 on either side of the insert arms are pressed inwardly toward the insert 14 and into contact with surfaces defining the recesses 174, at or near the surfaces 175, prohibiting further rotation of the insert 14 about the axis B with respect to the receiver 10. The receiver 10, retainer 12 and insert 14 combination is now in a desired pre-assembled state and ready for assembly with the shank 4 either at the factory, by surgery staff prior to implantation, or directly upon an implanted shank 4 as will be described herein.

The bone screw shank 4 or an entire assembly 1 made up of the assembled shank 4, receiver 10, retainer 12 and compression insert 14, is screwed into a bone, such as the vertebra 17 shown in phantom in FIG. 23, by rotation of the shank 4 using a suitable driving tool that operably drives and rotates the shank body 6 by engagement thereof at the internal drive 46. Specifically, the vertebra 17 may be pre-drilled to minimize stressing the bone and have a guide wire (not shown) inserted therein to provide a guide for the placement and angle of the shank 4 with respect to the vertebra. A further tap hole may be made using a tap with the guide wire as a guide. Then, the bone screw shank 4 or the entire assembly 1 is threaded onto the guide wire utilizing the cannulation bore 50 by first threading the wire into the opening at the bottom 25 and then out of the top opening at the drive feature 46. The shank 4 is then driven into the vertebra using the wire as a placement guide. It is foreseen that the shank and other bone screw assembly parts, the rod 21 (also having a central lumen in some embodiments) and the closure top 18 (also with a central bore) can be inserted in a percutaneous or minimally invasive surgical manner, utilizing guide wires and attachable tower tools mating with the receiver. When the shank 4 is driven into the vertebra 17 without the remainder of the assembly 1, the shank 4 may either be driven to a desired final location or may be driven to a location slightly above or proud to provide for ease in assembly with the pre-assembled receiver, compression insert and retainer.

With reference to FIGS. 23-28, whether it is desired for the shank 4 to be “popped” on to the receiver pre-assembly (receiver 10, retainer 12 and insert 14) before or after implantation of the shank 4 into bone, the following steps apply: With reference to FIG. 23, the receiver 10 is placed over the shank head 8 top surface 38 and the shank is “popped” into the receiver by pushing the shank head 8 into the receiver opening 110 and the shank surface 36 into contact with the retainer frusto-conical surface 145. With reference to FIG. 25, the retainer 12 and shank head 8 are then moved further into the receiver cavity 61 defined by the cylindrical surface 98 with the shank head hemisphere 40 clearing the edge or surface 106 defining the most narrow part of the receiver opening 110. Also, as shown in FIG. 24, at this time, the shank head 8 has pushed the retainer top surface 126 into abutment with the insert bottom surface 162. With reference to FIG. 25, as the shank head 8 continues to push upwardly into the bore 141 of the retainer 12 as well as into the through bore 180 of the insert 14, the retainer 12 begins to expand outwardly toward the receiver cylindrical surface 98. FIG. 25 shows maximum expansion of the retainer 12 about the shank head 8 with upward movement of the retainer 12 being blocked by the insert 14 that in turn is blocked from upward movement by the insert top surfaces 160 abutting against the receiver guide and advancement structures 72. FIG. 26 illustrates full capture of the shank head 8 by the retainer 12. The hemisphere 40 of the shank head 8 is now located entirely above the retainer 12 with the shank surface 36 in engagement with the retainer cylindrical surface 147. Also, the insert collet 15 inner surface 188 is now in frictional engagement with the shank surface 36 located above the hemisphere 40 and the shank surface 36 located near the top surface 38 of the shank upper portion 8 is in engagement with some of the ridges of the shank gripping portion 190.

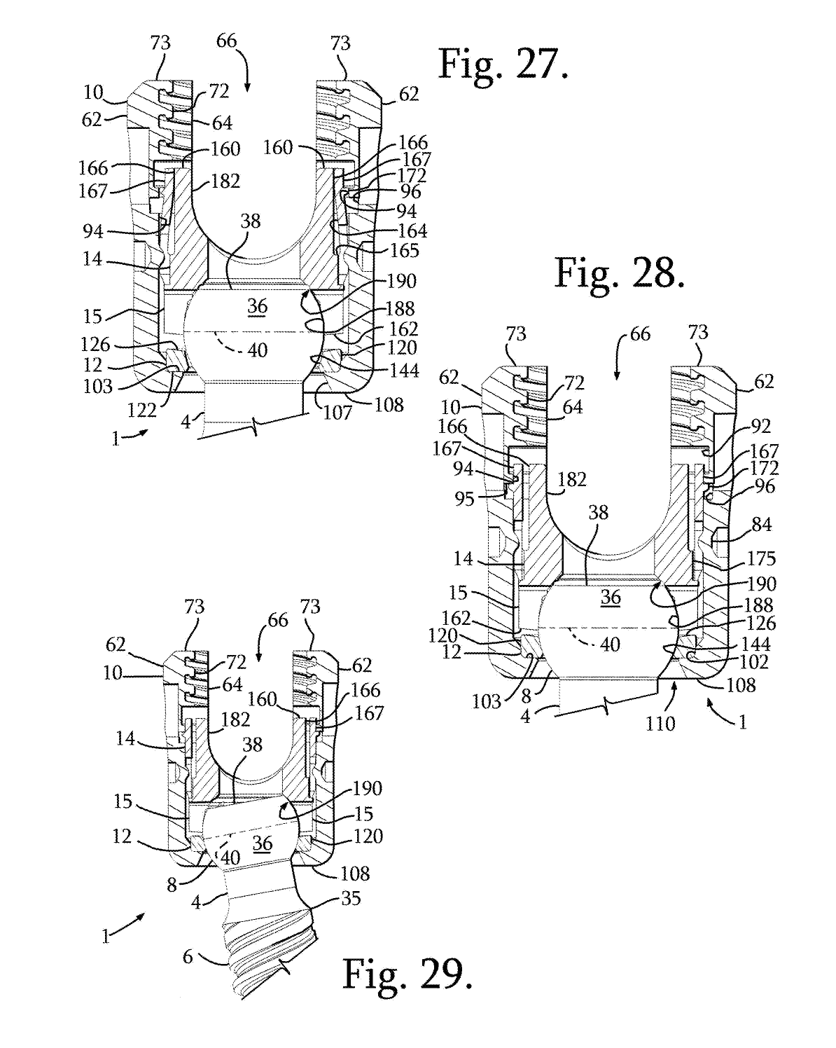

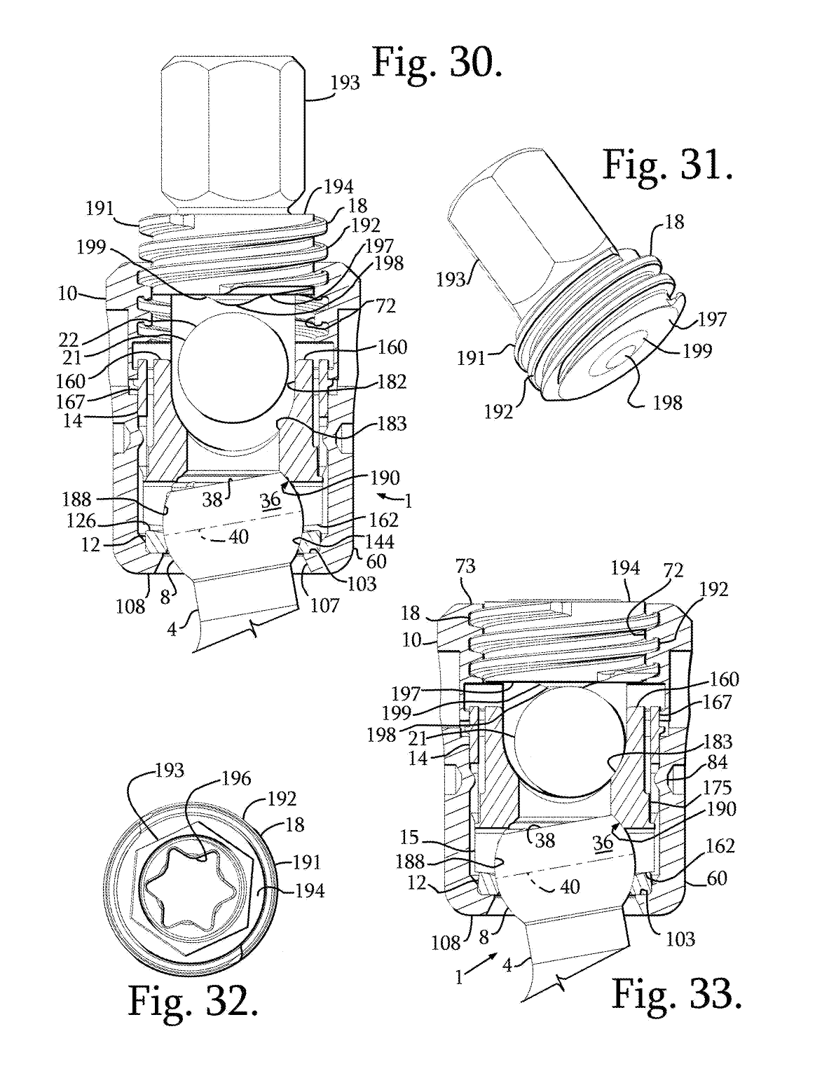

With reference to FIG. 27, to seat the retainer 12 on the seating surface 103 of the receiver 10, the receiver 10 is pulled up and away from the shank 4 (or, if the shank 4 is not implanted in bone, both the shank 4 and the receiver 10 may be grasped and pulled away from one another). As the retainer 12 and shank 4 are repositioned in a lower portion of the cavity 61, the insert 14 (now frictionally engaged to the shank head 8 at the collet 15) is also moved downwardly with the resilient arm outer portions 167 being pressed inwardly toward the axis B and toward the arm inner portions 166, the lips 172 clearing the cylindrical surfaces 94 until the arm outer portions 167 are returned to a substantially neutral position as shown in FIG. 28 with the lips 172 now received by a receiver portion defined by the annular surface 95 and the cylindrical surface 96. At this time, the annular surface 95 prohibits upward movement of the insert 14 at the lips 172 and thus helps to maintain the insert collet surfaces 188 and 190 in frictional or friction fit engagement with the shank head surface 36 while allowing pivoting of the shank 4 with respect to the receiver 10 when some force is applied to the shank 4 or to the receiver 10 to place the shank and receiver into a desired angular orientation with one another, for example, as shown in FIG. 29. At this time, the shank and receiver may be placed in a variety of angular orientations with respect to one another, using some force, and such orientation will be maintained by the friction fit relationship between the shank head 8 and the collet 15 portion of the insert 14. Thus, desirable, non-floppy angular adjustments may be made during surgery. Also with respect to FIGS. 27 and 28, it is noted that downward movement of the insert 14 with respect to the receiver 10 is possible because the crimped wall 84 can be moved within the vertically oblong insert recess 174. However, undesirable rotational movement of the insert 14 with respect to the receiver 10 is prohibited by the crimped wall portion 84 abutting against the more closely spaced vertical walls defining the recess 174.

With reference to FIGS. 30 and 33, the assembly 1 as shown in FIG. 29 is further shown being assembled with a 5.5 millimeter rod 21 and the closure top 18 previously described herein. The closure top 18 is driven into the receiver guide and advancement structure 72 using a socket type driver (not shown) that receives the break-off head 193. As the driver is rotated, the closure top 18 guide and advancement structure 192 is fully mated with the receiver guide and advancement structure 72 causing downward movement of the closure top 18 onto the rod 21, the rod in turn pressing downwardly on the insert 14, pressing the insert deeper into the receiver 10, locking the insert 14 against the shank head 8 which is now no longer pivotable with respect to the receiver 10. As stated above, the closure head 193 will twist or break off at a desired torque at which time the rod 21 will be frictionally engaging both the closure 18 and the insert 14 and the insert 14 will be in locked frictional engagement with the shank head 8, the shank head 8 pressing the retainer downwardly against the receiver seat 103 and outwardly against the receiver cylindrical surface 102.

With reference to FIGS. 33 and 33A, if the retainer 12 is made from a resilient material, such as a titanium alloy or stainless steel, and if the retainer 12 includes a plurality of outer slots or notches 128 as shown in the illustrated retainer 12, as the shank head 8 pressed downwardly and outwardly against the retainer surfaces 143 and 144, there may be a tendency of the retainer 12 to fold or move inwardly and upwardly along and toward the shank 8 surface 36 as shown in FIG. 33A, causing an upper portion of the retainer outer surface 120 to be pulled or otherwise maneuvered or moved slightly away from the receiver cylindrical surface 102. Expansion locking of the retainer 12 against the surface 102 is not compromised by such a “folding in” of the retainer toward the shank head 8 as the retainer 12 bottom surface 122 is retained in locked frictional engagement with the receiver seating surface 103 that is sized and shaped to support a substantial portion of the retainer at the bottom surface 122. As is shown in FIG. 33A, the “folding in” in such an embodiment is minor with the retainer corner surfaces 127 still closely held at or near the receiver corner surfaces 105.

It is noted that when the retainer 12 is made from a material that is harder than the material used for the receiver 10 and shank 4, such as when the receiver and shank are made from a titanium alloy and the retainer 12 is made from a cobalt-chrome alloy, the “folding in” exhibited in FIG. 33A does not occur, even when there are a plurality of notches in the retainer. The retainer outer surface 120 remains in full contact with the receiver cylindrical surface 102 during locking of the shank head 8 against the retainer 12. Furthermore, when the insert 14 is made from a harder material than the shank 4, for example, when the insert 14 is made from a cobalt-chrome alloy and the shank is made from titanium, titanium alloy or stainless steal, the insert gripping portion 190 advantageously digs into the shank head 8 more deeply during locking than when both the insert 14 and the shank 4 are made from the same material.

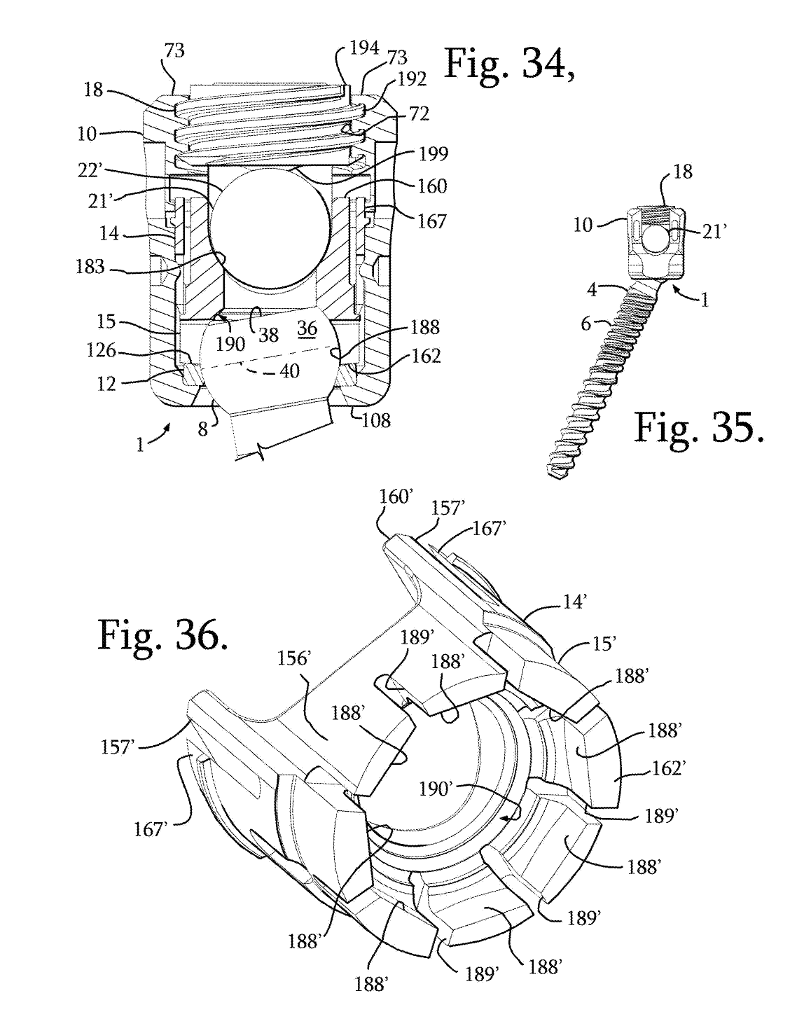

With further reference to FIG. 33 and also with reference to FIG. 34, it is noted that the closure 18 with lower nub 198 advantageously cooperates with rods or other longitudinal connecting members having various diameters. As is shown in FIGS. 30 and 33, when a rod having a 5.5 mm diameter is used with the assembly 1 and the closure 18, the rod 21 is loosely received by the arms 157 of the insert 14 at the saddle surfaces 182 and 183. However, as the closure 18 presses downwardly on the rod 21, the nub 198 presses the rod 21 in a lateral direction against one arm 157 more than the opposite arm, sufficiently securing the rod between the insert 14 wall and the nub 198. With reference to FIG. 34, when a 6 mm rod 21′ is used with the insert 14 and the closure 18, the rod 21′ is more closely received within the insert saddle 182 and the closure nub 198 presses firmly and centrally on the rod 21′. FIG. 35 illustrates the assembly 1 with the rod 21′ and closure 18 wherein the shank 4 is pivoted at a twenty-six degree angle (cephalic) with respect to the shank 10.

It is noted that if the surgeon wishes to further manipulate the rod 21 or 21′ or remove the rod, the closure top 18 may be loosened (and removed of desired) by using a driver in the closure drive 196 to rotate the closure 18 and move the closure 18 in an upward direction away from the rod 21 or 21′. At such time, the receiver 10 can again be tilted or otherwise angularly manipulated with respect to the shank 4 in a non-floppy manner using some force.

FIG. 36 illustrates an alternative insert 14′ for use in place of the insert 14 in the assembly 1 shown in FIGS. 1-35. The insert 14′ is substantially identical in form and function to the insert 14 with the exception of a plurality of planar or flat inner surfaces 188′ that replace the collet 15 radiused surface 188. The insert 14′ is thus assembled with the other bone screw components 10, 12 and 4 in a manner identical to what is described previously herein with respect to the insert 14. Also, for example, the insert 14′ includes a lower friction fit collet portion 15′, a body 156′, upstanding arms 157′, arm top surfaces 160′, collet bottom surfaces 162′, arm outer resilient surface portions 167′, lower collet slots 189′ and a shank gripping portion 190′ that are substantially the same or similar to the respective lower friction fit collet portion 15, body 156, upstanding arms 157, arm top surfaces 160, collet bottom surfaces 162, arm outer resilient surface portions 167, lower collet slots 189 and shank gripping portion 190 previously described herein with respect to the insert 14, as well as the other features previously discussed herein with respect to the insert 14. Although the inner surfaces 188′ are planar, such surfaces resiliently press against the shank head 8 at the surface 36 during manipulation of the shank 4 with respect to the receiver 10 to provide a non-floppy, friction fit between the insert 14′ and the shank head 8 that allows for movement of the shank 4 with respect to the insert 14′ when some force is used to pivot the shank 4 with respect to the receiver 10 during a surgical procedure prior to locking of the insert 14′ gripping portion 190′ against the shank head 8.

It is noted that polyaxial bone screw assemblies 1 (and 201 and 401 described below) according to embodiments of the invention may be used with longitudinal connecting member assemblies that are sometimes called “soft” or “dynamic” connectors that may include one or more sleeves, as described, for example, in applicants' patent application U.S. Ser. No. 13/573,516 filed Sep. 19, 2012, and incorporated by reference herein. Such assemblies may have sleeves with varied lengths of tubular extensions on one or both sides thereof and further cooperate with an inner tensioned cord, one or more bumpers, one or more spacers and one or more fixers or blockers for fixing the cord to the connector assembly without fixing the cord directly to a bone anchor. A variety of such connector components are also described in Applicants' U.S. patent application Ser. No. 12/802,849 filed Jun. 15, 2010 (U.S. Publication No. 2010/0331887), also incorporated by reference herein.

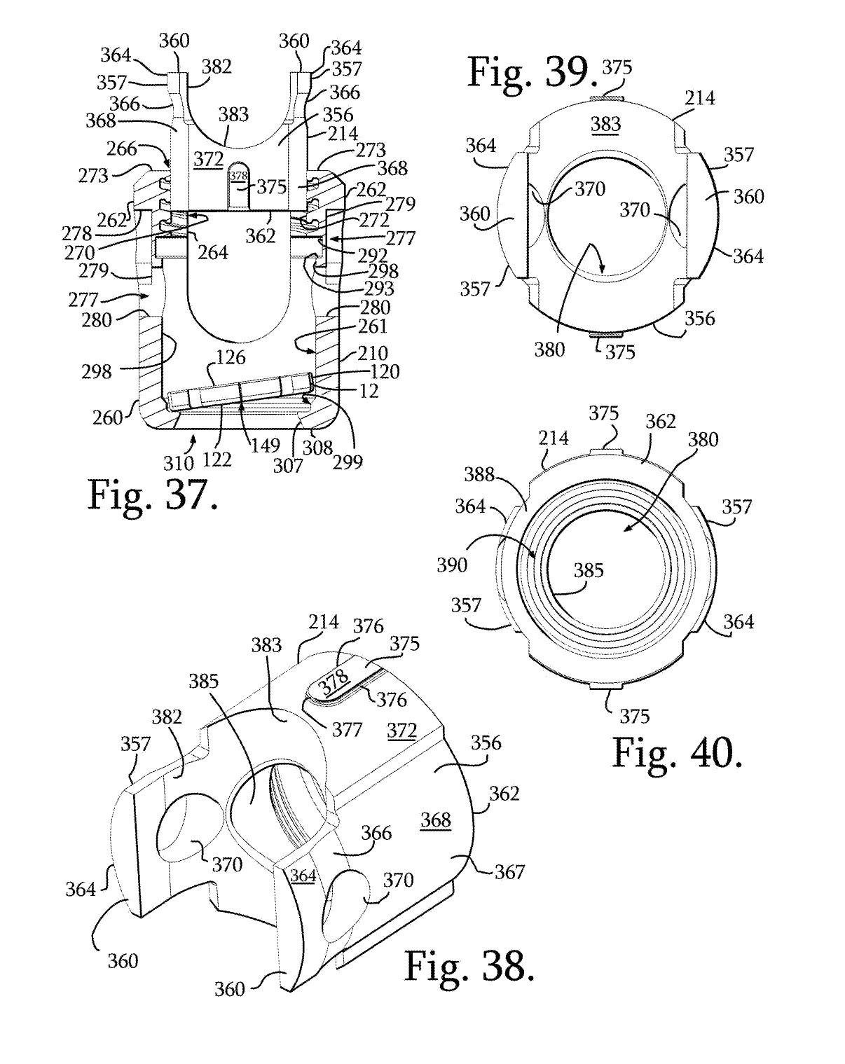

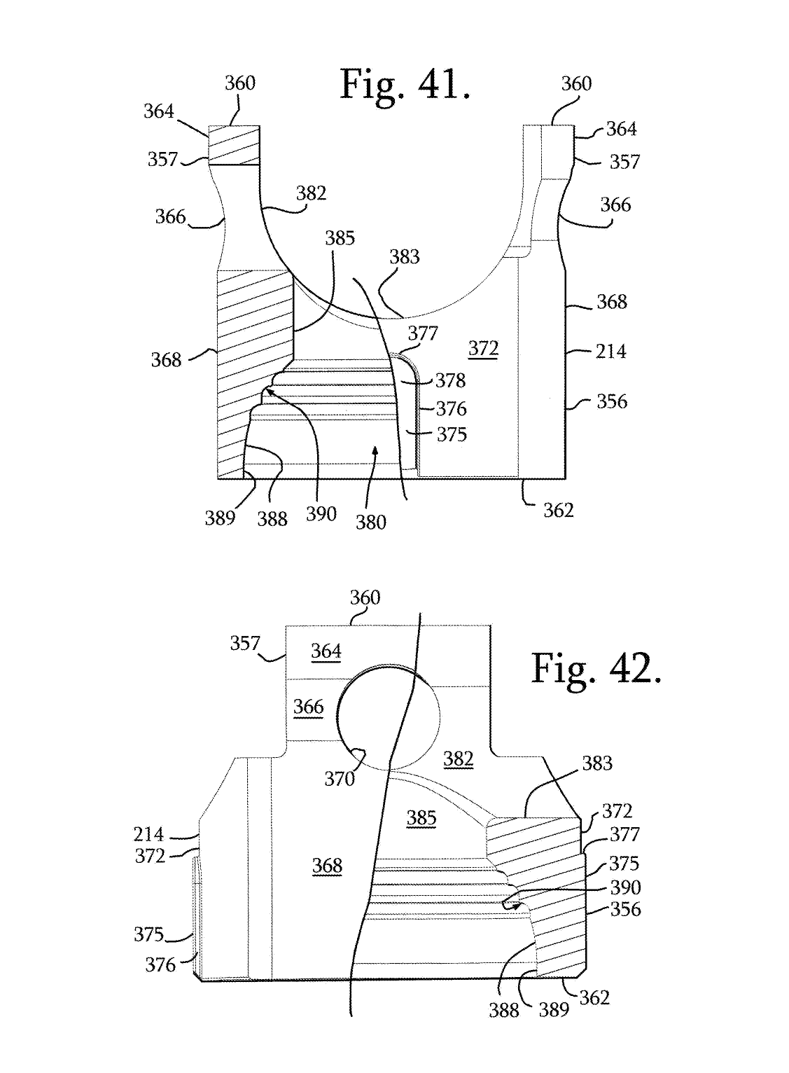

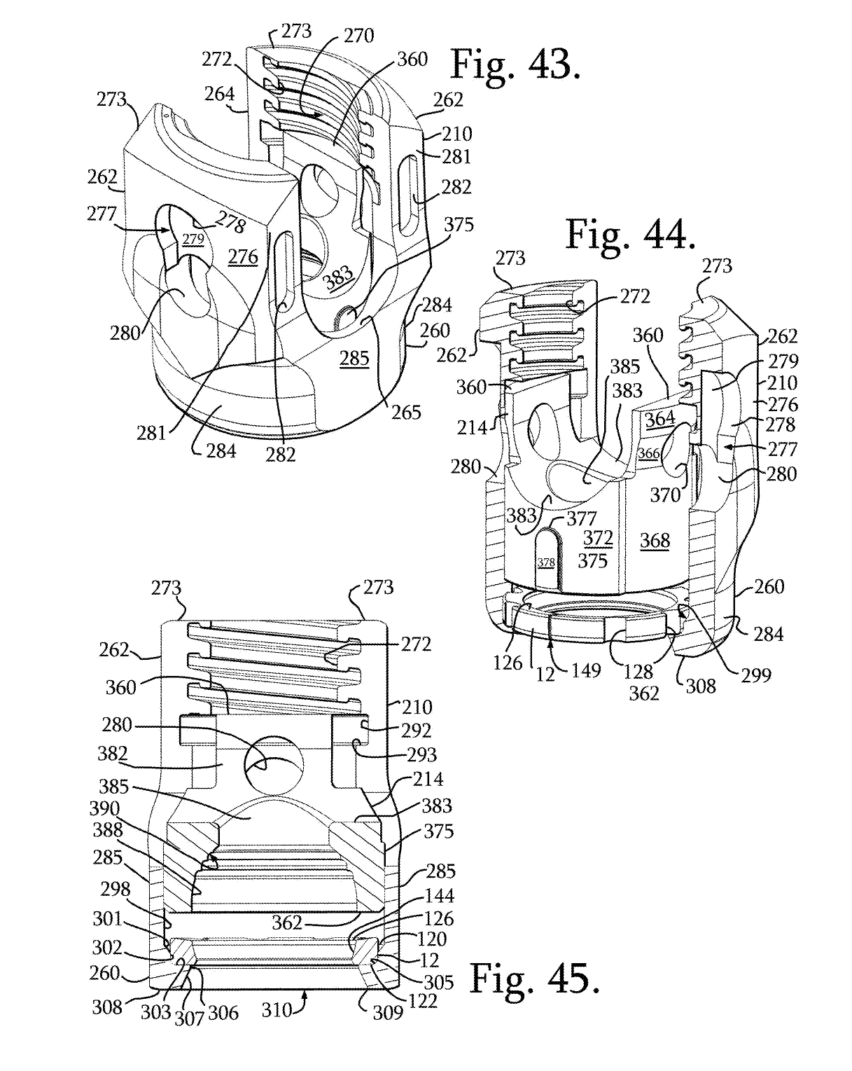

With reference to FIGS. 37-56, the reference number 201 generally represents an embodiment of an alternative bone anchor assembly the includes the shank 4 and the retainer 12 of the assembly 1 and replaces the receiver 10 with a receiver 210 and replaces the insert 14 with an insert 214. In operation, the insert 214 advantageously frictionally engages the bone screw shank upper portion 8 as well as engaging the receiver 210 in a diametric interference fit engagement, the insert initially pressing down on the shank upper portion just enough to provide a movable friction fit and then pressing further to ultimately locking the shank 4 in a desired angular position with respect to the receiver 210, the frictional locking between the insert 214 and the receiver 210 occurring at a location spaced from the receiver 210 upstanding arms, thus avoiding undesirable outward splay of the receiver arms. The insert 214 retains such locked position even if, for example, a rod and closure are later removed and the rod is replaced with another rod or other longitudinal connecting member or member component. At such time, the receiver 210 cannot be tilted or otherwise angularly manipulated with respect to the shank 4. Thus, the assembly 201 can advantageously perform like a strong, mono-axial screw, regardless of the orientation of the shank 4 with respect to the receiver 210.

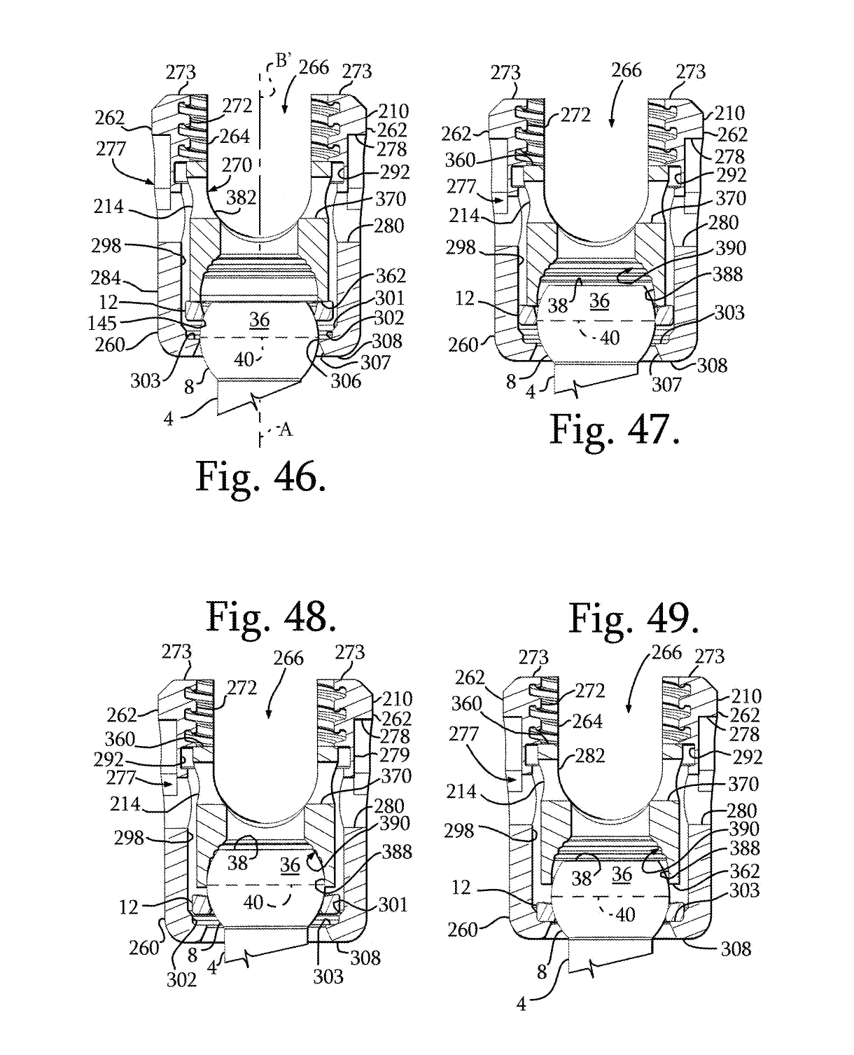

With reference to FIGS. 37 and 43-46, the receiver 210 has a generally U-shaped appearance with partially discontinuous cylindrical inner and outer profiles as well as planar and other curved surfaces. The receiver 10 has an axis of rotation B′ that is shown, for example, in FIG. 46 as being aligned with and the same as the axis of rotation A of the shank 4, such orientation being desirable, but not required during assembly of the receiver 210 with the shank 4. After the receiver 210 is pivotally attached to the shank 4, either before or after the shank 4 is implanted in the vertebra 17, the axis B′ is typically disposed at an angle with respect to the axis A.

The receiver 210 includes a base 260 forming an inner cavity, generally 261. Two opposed arms 262 extend upwardly from the base 260 and form a U-shaped channel 264 defined in part by a lower rod receiving portion 265 and having an upper opening 266. Other features of the receiver 210 include, but are not limited to inner receiver arms surfaces, generally 270 that include a guide and advancement structure 272 located near arm top surfaces 273. In the illustrated embodiment, the guide and advancement structure 272 is a partial helically wound interlocking flangeform configured to mate under rotation with a similar structure on the closure structure 18 as previously described herein with respect to the receiver 10. However, it is foreseen that for certain embodiments of the invention, the guide and advancement structure 272 could alternatively be a square-shaped thread, a buttress thread, a reverse angle thread or other thread-like or non-thread-like helically wound discontinuous advancement structures, for operably guiding under rotation and advancing the closure structure 18 downward between the arms 262, as well as eventual torquing when the closure structure 18 abuts against the rod 21, 21′ or other longitudinal connecting member. It is foreseen that the arms 262 could have break-off extensions.

Each arm 262 has an outer surface 76 with one or more tool receiving grooves, recesses or apertures. In the illustrated embodiment a centrally located tool receiving recess, generally 277, includes an upper recessed portion defined in part by a partially cylindrical wall 278 and a back wall 279 that further communicates with a lower through bore 280. The recess is centrally located between arm side surfaces 281 and near, but spaced from the top surface 273. Each side surface 281 also has an oblong recess 282 (total of four recesses) that may be used to receive portions of manipulating tools. The recesses 282 do not extend all the way through the respective arm portions. The through bore 280 does extend completely through the arms 262. Opposed receiver base portions 284 are located directly beneath each receiver arm 262 and are each substantially cylindrical in form. Located between each base portion 284 and also generally beneath each arm side surface 281 are opposed flat or planar base portions 285. The portions 285 also partially define the receiver lower seating portion 265.

With particular reference to FIGS. 37 and 45, returning to the interior surface 270 of the receiver arms 262, located below the guide and advancement structure 272 is a discontinuous cylindrical surface 292 partially defining a run-out feature for the guide and advancement structure 272. The cylindrical surface 292 is sized and shaped to receive an upper portion of the insert 14. Therefore, the surface 292 has a diameter greater than a greater diameter of the guide and advancement structure 272. The receiver arms may further includes sloped, stepped or chamfered surfaces above and below the surface 292. Directly below the surface 292 is a lip or ledge surface 293 that extends inwardly towards the axis B. The ledge 293 extends from the cylindrical surface 292 inwardly to a cylindrical surface 298 that is discontinuous at the arms 262 and continuous at the base 260. The surface 298 thus defines lower portions of the receiver inner arms 270 as well as a portion of the base cavity 261. The cylindrical surface 298 is also parallel to the axis B′ and has a diameter that is smaller than the diameter of the surface 292. The diameter of the surface 298 is sized and shaped to allow for expansion of the retainer 12 about the shank upper portion 8 within the receiver cavity 261. The surface 298 terminates at a lower stepped or tiered retainer seating and expansion locking portion, generally 299 that includes a substantially frusto-conical surface 301 adjacent the surface 298, a cylindrical surface 302, a bottom annular and planar seating surface 303, a rounded or radiused corner portion 305 connecting the surface 302 with the surface 303, a lower flared or tapered surface 307 opening to a bottom exterior surface 308 at a bottom opening, generally 310 of the receiver. The seating surface 303 terminates at a narrow cylindrical surface 306 that connects the seating surface 303 with the tapered surface 307. The surface 306 is substantially parallel to the axis B′ and has a diameter smaller than a diameter of the surface 302, the surface 302 also being substantially parallel to the axis B′. The surface 306 diameter is also smaller than a diameter of a lower opening edge 309 formed at the intersection of the surface 307 and the surface 308. It is noted that additional curved or radiused surfaces may be included in the seating portion 299 to provide for a graduated transition from the expansion chamber defined by the surface 298 to the retainer seating surface 303.

With particular reference to FIGS. 37-42, the compression insert 214 is illustrated that is sized and shaped to be received by and down-loaded into the receiver 210 at the upper opening 266. The compression insert 214 has an operational central axis that is the same as the central axis B′ of the receiver 210. Features of the insert 214 include a body 356 integral with a pair of upstanding arms 357. The body 356 is substantially cylindrical in outer appearance. Substantially planar arm top surfaces 360 are located opposite a bottom surface 362 of the body 356. Each of the arms 357 includes an upper outer outwardly flared surface portion 364 adjacent the top surface 360 that extends radially outwardly from the body 356 portion located directly below the arms 357. Located below each flared surface portion 364 is a curved, slightly, concave arm surface portion 366 that terminates at a cylindrical surface portion 368. The surface portion 368 extends downwardly along the insert body 356 and terminates at the bottom surface 362. A radius of the insert body 356 at the surface portion 368 measured from the axis B′ is smaller than a radius measured from the axis B′ to either of the flared arm surface portions 364. Each arm 357 further includes a circular through bore 370 formed therethrough that is centrally located at the arm portion 366 and has an upper portion extending through the flared surface portion 364 and a lower portion extending through the cylindrical surface 368. The through bores 370 are positioned opposite one another and run perpendicular to the axis B′.

At the insert body 356 located between each cylindrical surface portion 368 is another cylindrical surface portion 372. Thus, there are a pair of opposed body portions 372. A radius of the surface portion 372 measured from the axis B′ is greater than the radius of the surface portion 368 also measured from the central axis B′. Each insert body portion 372 terminates at the saddle 383 and also terminates at the insert bottom surface 362. Opposed narrow interference fit strips or tabs 375 are centrally located on the surfaces 372, each extending outwardly from the respective surface 372. In the illustrated embodiment, the strips 375 are integral with the insert surface 372. Each strip 375 is elongate, having opposed parallel side surfaces 376 and extending from a rounded upper surface 377 located near the saddle surface 383 to a location at or near the bottom surface 362. Each strip 375 runs substantially parallel to the axis B′. Each strip has a curved, partially cylindrical outer surface 378. A diameter measured between the surfaces 378 is greater than a diameter of the insert body 356 measured between the opposing cylindrical surface portions 372. Furthermore, the insert 214 is sized and shaped so that the diameter measured between surfaces 372 is less than a diameter of the receiver 210 measured at the expansion chamber defined by the surface 298 and the diameter measured between strip surfaces 378 is slightly greater than the expansion chamber diameter defined by the surface 298. The interference strips 375 are located centrally on the surfaces 372 so that the strips 375 ultimately engage the receiver 210 at the receiver surface 298 located near the base surfaces 285 that are located substantially centrally between the arms 262 and beneath the surface 268. Thus, during friction fit manipulation of the assembly 201 when the bone screw shank 4 is pivoted with respect to the receiver 210 using some force (so in a non-floppy manner) and also during final locking of the polyaxial mechanism of the assembly 201, as the insert 214 is pressed downwardly against both the shank head 8 and the receiver 210, a diametric interference fit occurs between the insert and the receiver that does not place an outward splaying force on the receiver arms 262.

Returning to the inner surfaces of the insert 214, a through bore, generally 380, is disposed primarily within and through the insert 214 and communicates with a generally U-shaped through channel formed by a saddle surface 382 that is substantially defined by the upstanding arms 357. Near the top surfaces 360, the saddle surface 382 is substantially planar. The saddle 382 has a lower seat 383 sized and shaped to closely, snugly engage the rod 21′ or other longitudinal connecting member. The interference strips 375 are located centrally below the seat 383. It is foreseen that an alternative embodiment may be configured to include planar holding surfaces that closely hold a square or rectangular bar as well as hold a cylindrical rod-shaped, cord, or sleeved cord longitudinal connecting member.

The bore, generally 380, is further defined by an inner cylindrical surface 385 that communicates with the seat 383 and a lower concave, radiused inner surface 388 that terminates at or near the bottom surface 362, the surface 388 having a radius or surface for closely receiving and frictionally engaging the surface 36 of the shank upper portion 8. In the illustrated embodiment, spanning between the surface 388 and the bottom surface 362 is a substantially cylindrical surface 389. At an upper portion thereof, the surface 388 terminates at a shank gripping surface portion, generally 390. The gripping surface portion 390 extends upwardly to the cylindrical surface 385. The gripping surface portion 390 includes more than one and up to a plurality of stepped surfaces or ridges sized and shaped to grip and penetrate into the shank head 8 when the insert 214 is finally locked against the head surface 36. The illustrated gripping portion 390 includes at least three ridges or edges. It is foreseen that the shank gripping surface portion 390 and also the surface 388 may additionally or alternatively include a roughened or textured surface or surface finish, or may be scored, knurled, or the like, for enhancing frictional engagement with the shank upper portion 8.

The compression insert 214 through bore 380 is sized and shaped to receive a driving tool therethrough that engages the shank drive feature 46 when the shank body 6 is driven into bone with the receiver 210 attached. Also, the bore may receive a manipulation tool used for releasing the insert 214 from a locked position with the 210 receiver, the tool pressing down on the shank and gripping the insert at tool engaging features 370. Each of the arms 357 and the insert body 356 may include more surface features, such as cut-outs notches, bevels, etc. to provide adequate clearance for inserting the insert 214 into the receiver and cooperating with the retainer 12 during the different assembly steps.

The insert body 356 has a diameter measured between the surfaces 368 that is slightly greater than a diameter between crests of the guide and advancement structure 272 of the receiver 210. As illustrated in FIG. 37, the insert 214 presses the receiver arms 262 outwardly during top loading of the compression insert 214 into the receiver opening 266. Thus, a desirable material for the receiver 210 is a more resilient material such as a titanium alloy, while a desirable material for the insert 214 is a harder material, such as cobalt-chrome. With reference to FIG. 43 Once the arms 357 of the insert 214 are generally located beneath the guide and advancement structure 272, the receiver arms 262 return to a neutral position with the insert arm flared upper portions 364 trapped below the structure 272 in the receiver groove formed by the cylindrical surface 292.

The assembly of the receiver 210 and retainer 12 is the same of similar to what has been described previously herein with respect to the assembly 1. The assembly of the insert 214 into the receiver 210, as described in the previous paragraph herein is shown, for example in FIGS. 37 and 43-45. As described above, the insert 214 is top loaded through the receiver opening 266 with the insert arms 357 aligned with the receiver arms 262. The insert 214 is initially pushed downwardly, with force, until the insert arm top surfaces 360 are located below the receiver guide and advancement structures 272. As the insert 214 is pressed downwardly, the interference fit strips 375 frictionally engage the receiver inner cylindrical surface 298, fixing the insert 214 in frictional engagement with the receiver 210 in a desired alignment, the receiver U-shaped channel defining surface 264 being aligned with the insert saddle surface 382. As illustrated in FIGS. 43-46, the insert 214 is also in a desired position within the receiver 210, capturing the retainer 12 in the receiver 210 and ready for assembly with the shank 4.

With reference to FIGS. 46-52, whether it is desired for the shank 4 to be “popped” on to the receiver pre-assembly (receiver 210, retainer 12 and insert 214) before or after implantation of the shank 4 into bone, the following steps apply: With reference to FIG. 46, the receiver 210 is placed over the shank head 8 top surface 38 and the shank is “popped” into the receiver by pushing the shank head 8 into the receiver opening 310 and the shank surface 36 into contact with the retainer frusto-conical surface 145. With further reference to FIG. 46, the retainer 12 and shank head 8 are then moved further into the receiver cavity 261 defined by the cylindrical surface 298 with the shank head hemisphere 40 clearing the edge or surface 306 defining the most narrow part of the receiver opening 310. Also, at this time, the shank head 8 has pushed the retainer top surface 126 into abutment with the insert bottom surface 362. With reference to FIG. 47, as the shank head 8 continues to push upwardly into the bore 141 of the retainer 12 as well as into the through bore 380 of the insert 214, the retainer 12 begins to expand outwardly toward the receiver cylindrical surface 298. FIG. 47 shows maximum expansion of the retainer 12 about the shank head 8 with upward movement of the retainer 12 being blocked by the insert 214. FIG. 48 illustrates full capture of the shank head 8 by the retainer 12. The hemisphere 40 of the shank head 8 is now located entirely above the retainer 12 with the shank surface 36 in engagement with one or more inner surfaces of the retainer 12. Also, the insert inner surfaces 388 and 390 are now engaging the shank surface 36 located above the hemisphere 40.

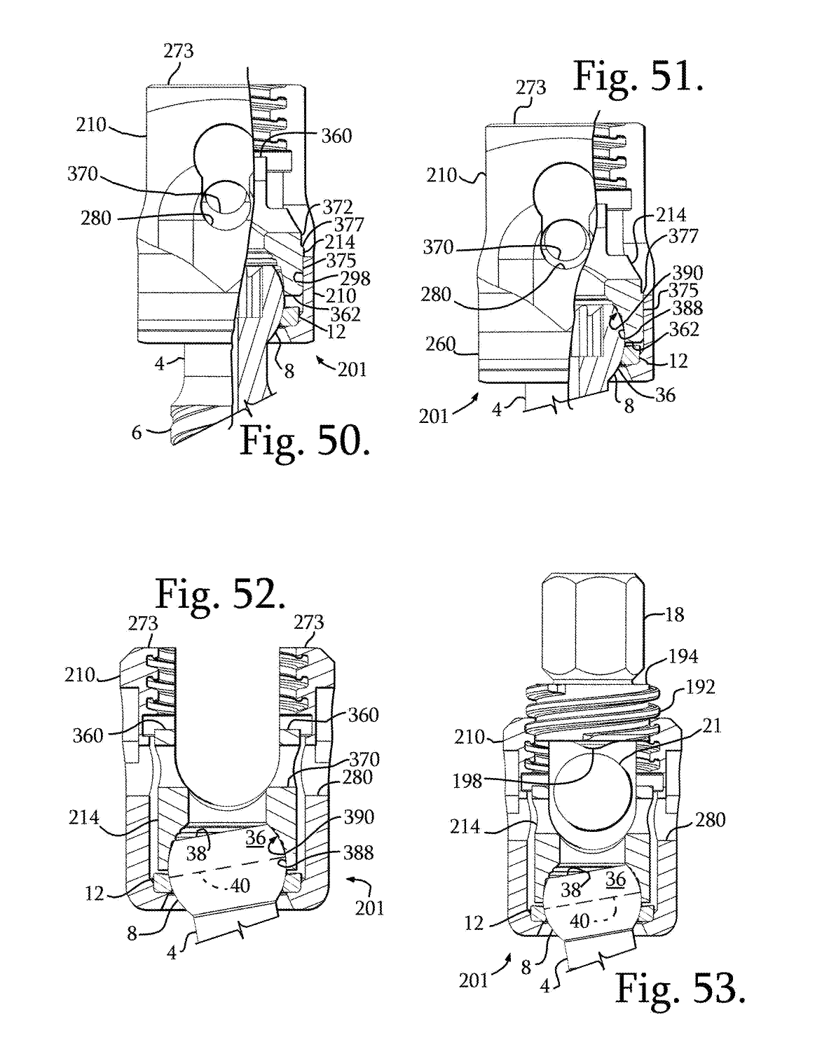

With reference to FIGS. 49 and 50, to seat the retainer 12 on the seating surface 303 of the receiver 210, the receiver 210 is pulled up and away from the shank 4 (or, if the shank 4 is not implanted in bone, both the shank 4 and the receiver 210 may be grasped and pulled away from one another). As the retainer 12 and shank 4 are repositioned in a lower portion of the cavity 261, the insert 214 and the shank head 8 also pull away from one another as the insert 214 is fixed to the receiver 210 at the interference fit strips 375, keeping the insert 214 in an upper portion of the expansion chamber defined by the receiver cylindrical surface 298. With reference to FIG. 51, the insert 14 is pressed downwardly with force to a desired “friction fit” location wherein the insert surfaces 388 and 390 are pressing on the shank head 8 outer surface 36 to an extent that the shank 4 can be moved in a non-floppy manner to a variety of angular orientations with respect to the receiver 210, as shown, for example, in FIG. 52. If the surgeon wishes to lock the polyaxial mechanism of the assembly 201, the insert 214 may be pressed downwardly further, either at the top surfaces 360 or with tooling extending through the receiver through bores 280 and the insert bores 370. Thereafter, a longitudinal connecting member, such as a rod and a closure mechanism, such as the closure 18 may be fixed to the assembly 201.

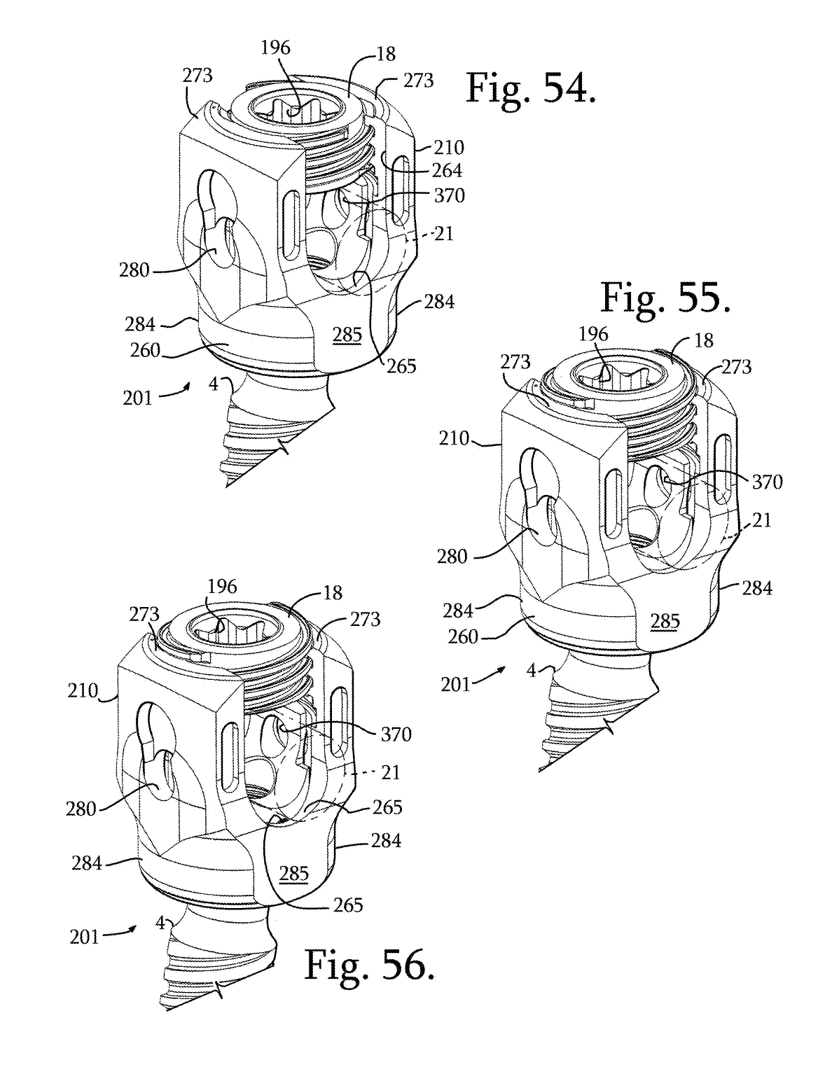

Alternatively, with reference to FIG. 53, the closure 18 previously described herein may be inserted between the receiver arms and rotated with the flange form 192 mating with the receiver flange form 272 to drive the closure downwardly into engagement with the rod 21, the rod 21 pressing the insert 214 down into locking engagement with the shank head 8. In a manner the same or similar to what has been previously described herein with respect to the assembly 1, the downward force of the shank head 8 presses the retainer 12 outwardly and downwardly into engagement with the receiver seating surfaces 302 and 303 to lock the shank 4 with respect to the receiver 210 as shown in FIG. 54.

With further reference to FIG. 54 and also FIGS. 55 and 56, if adjustment of the rod 21 (shown in phantom) is desired, a driving tool may be used to engage and rotate the closure 18 at the drive 196 and loosen the closure 18 as shown in FIG. 55. Thereafter, the rod may be manipulated without loosening the frictional engagement between the receiver 210 and the insert 214 and thus without loosening the locked angular position of the shank 4 with respect to the receiver 210 as the insert 214 will maintain a constant force on the shank head 8. With reference to FIG. 56, to return to a friction fit engagement between the shank head 8 and the insert 214 wherein the shank 4 may be pivoted with respect to the receiver 210, tooling may be used through the receiver through bores 280 to engage the insert 214 at the bores 370 and move the insert 214 upwardly within the receiver chamber as indicated by the portion of the interference strip 375 visible in FIG. 56. Thereafter, both the angle of the shank 4 with respect to the receiver 210 and the position of the rod 21 may be manipulated until a desired orientation is accomplished and the driving tool may be used at the closure drive 196 to rotate the closure 18 and press the insert 214 downwardly into locking engagement with the shank head 8.

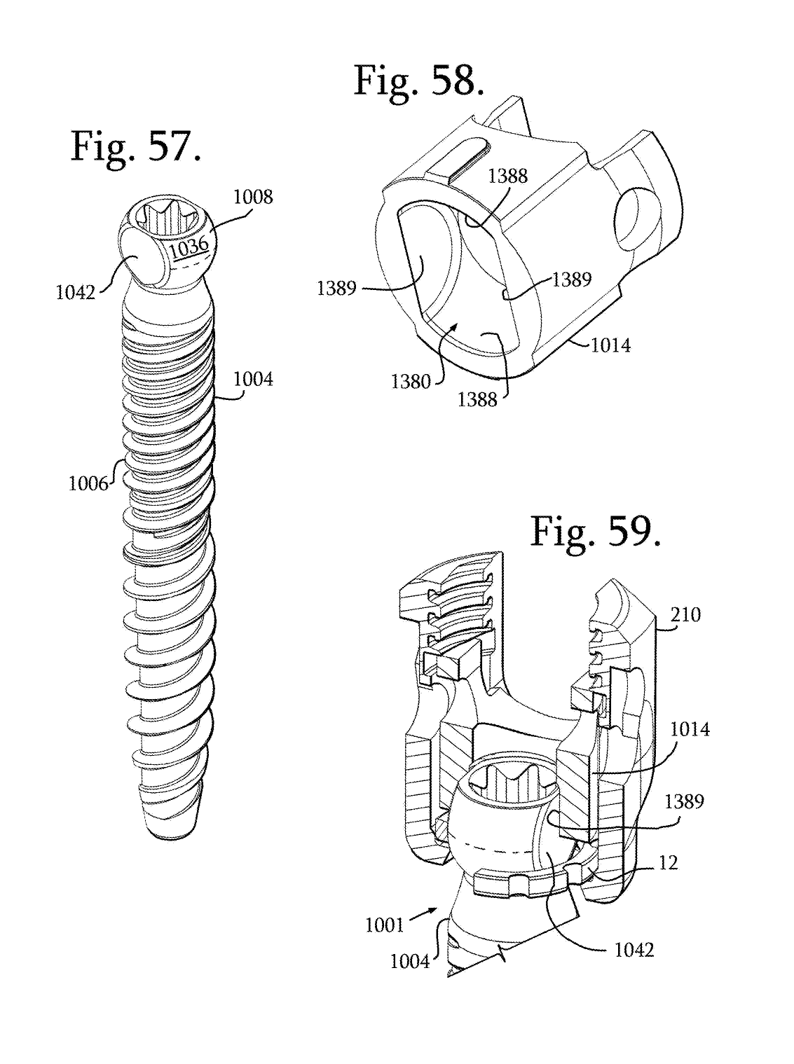

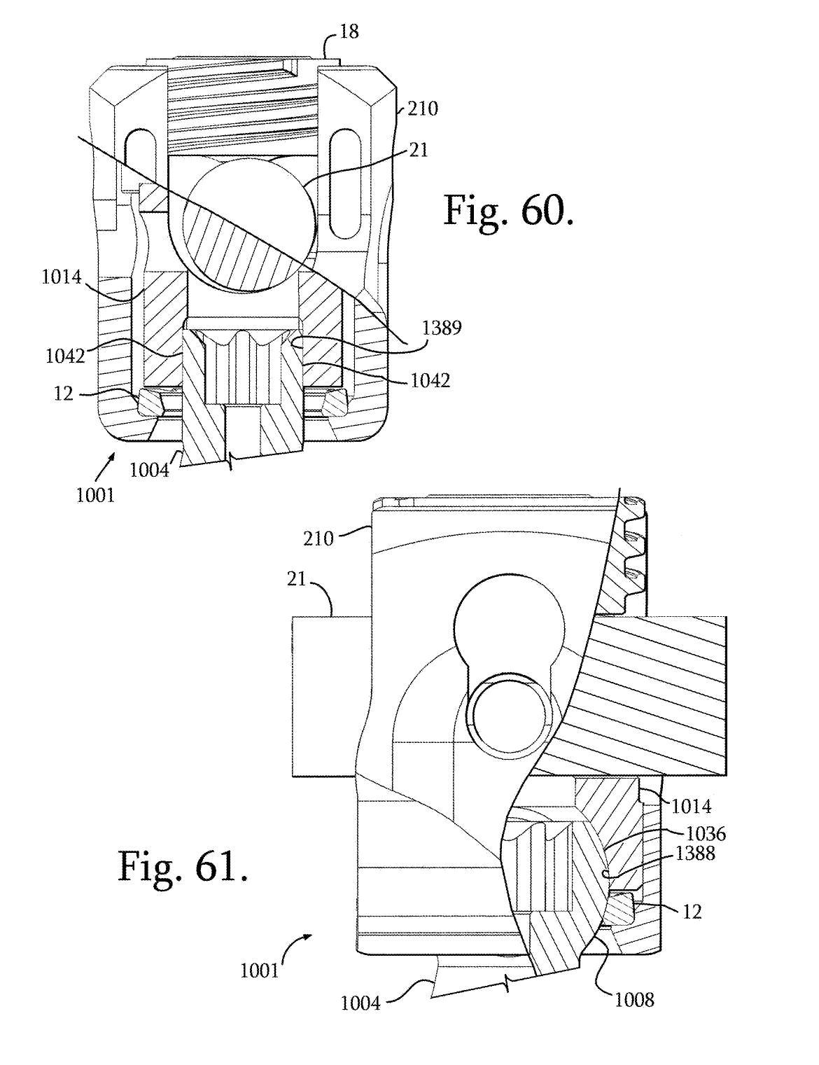

With reference to FIGS. 57-62, the reference number 1001 generally represents an alternative, uni-planar bone screw apparatus or assembly according to an embodiment of the invention. The assembly 1001 includes a shank 1004 substantially similar to the shank 4 previously described herein; the receiver 210 previously described herein; the retainer 12 previously described herein; and a locking friction fit pressure insert 1014 that is substantially similar to the insert 214 previously described herein. With particular reference to FIGS. 57 and 59, the uni-planar shank 1004 includes a body 1006 and a substantially spherical head 1008 the same or similar to the shank 4 body 6 and head 8 previously described herein with the exception that formed in a spherical head portion 1036 of the shank head 1008 are opposed and parallel flat planar surfaces 1042.

With reference to FIG. 58, the uni-planar locking insert 1014 is substantially similar to the insert 214 in form and function with the exception that a through bore, generally 1380, is sized and shaped to received the shank head 1008 and thus has opposed radiused surfaces 1388 for receiving and engaging the shank surfaces 1036 and opposed planar surfaces 1389 for receiving the shank planar surfaces 1042.

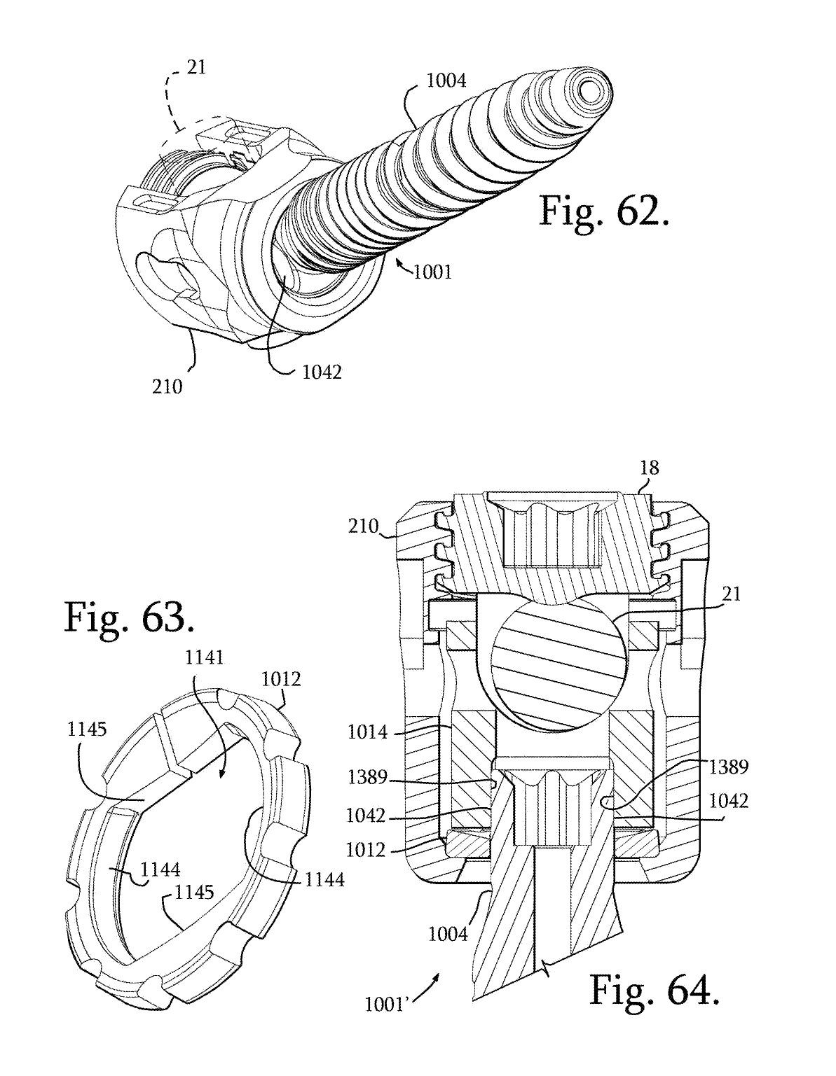

With reference to FIGS. 59 and 62, the retainer 12 and insert 1014 are loaded into the receiver 210 in a manner similar to that described previously herein with respect to the assembly 201. FIG. 59 illustrates the “popping” on of the uni-planar shank 1004 to the now mounted uni-planar insert 1014. The shank must be positioned such that the shank flat surfaces 1042 slide up along the insert flat surfaces 1389. Once the shank head 1008 passes through the retainer 12 and is captured thereby, the planar side surfaces 1042 are slidable along the insert surfaces 1389, allowing for articulation of the shank 1004 with respect to the receiver 210 in only one plane. Due to the fact that the insert 1014 is frictionally locked against the receiver 210, the single plane of articulation is in direct alignment with the length of the rod 21, shown for example, in FIG. 62. All of the other implantation and shank manipulation, friction fit and locking steps previously described herein with respect to the assembly 201 also apply to the assembly 1001.

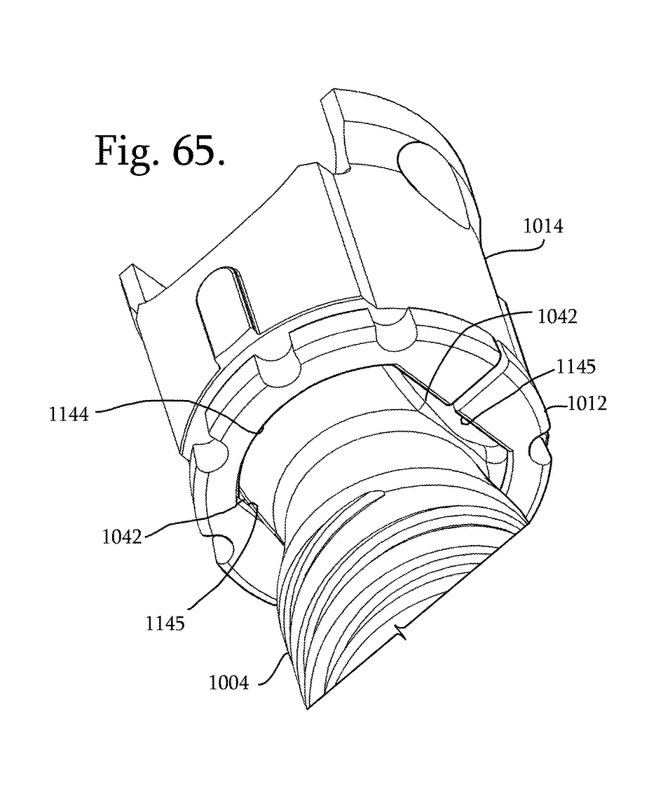

FIGS. 63-65 illustrate another embodiment, generally 1001′ that replaces the retainer 12 with a retainer 1012. The retainer 1012 is substantially similar to the retainer 12 in form and function with the exception that a bore 1141 is defined by opposed radiused surfaces 1144 and opposed planar surfaces 1145, the surfaces 1145 sized and shaped for receiving the planar surfaces 1042 of the shank 1004 as best shown in FIG. 65.

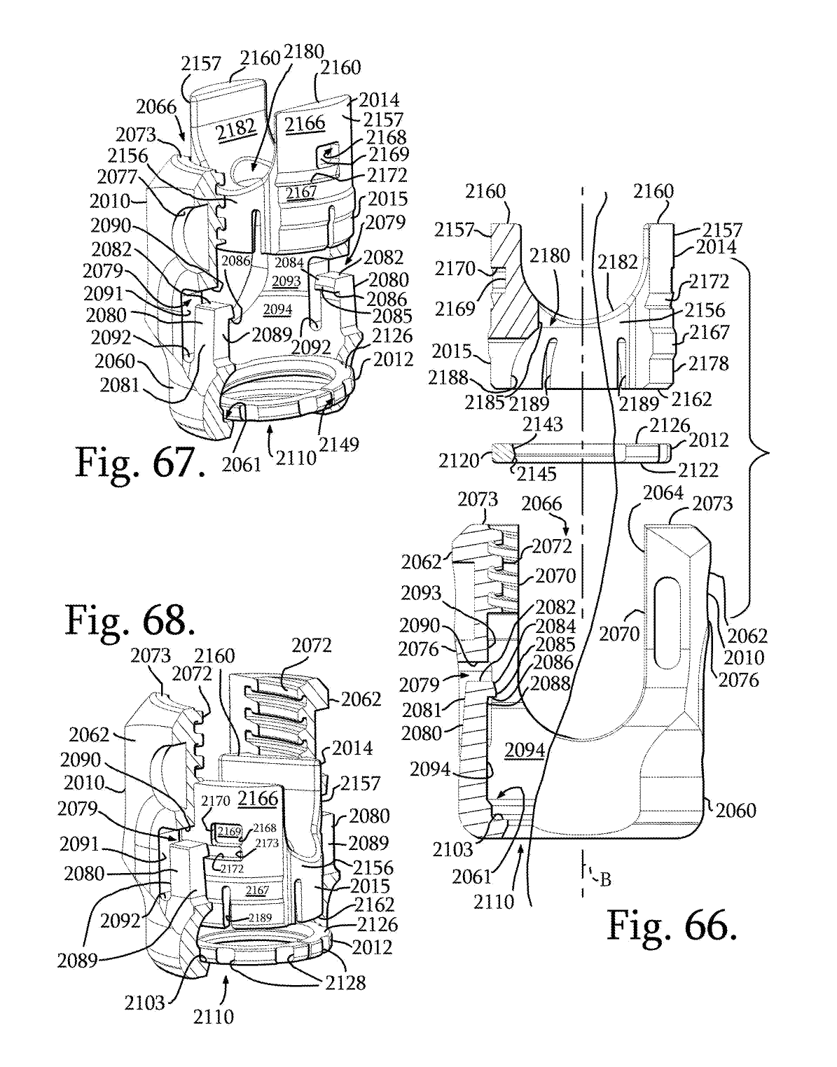

With reference to FIGS. 66-77, an alternative polyaxial bone screw assembly 2001 according to an embodiment of the invention is substantially similar to the assembly 1 previously described herein but with a receiver 2010 replacing the receiver 10 and an insert 2014 replacing the insert 14. Briefly, the insert 2014 includes a lower friction fit collet 2015 that is identical or substantially similar to the lower collet 15 of the insert 14, but the insert 2014 does not include resilient outer arm portions that cooperate with inner annular surfaces of the receiver to position the insert at desired locations within the receiver during various steps of assembly and operation thereof. Rather, the receiver 2010 now includes resilient inwardly facing arm portions or tabs that engage and cooperate with outer surfaces of the insert 2014 to result in a desired insert placement with respect to the receiver.

Specifically, the assembly 2001 includes a shank 2004 having a shank body 2006 and an integral upper portion or head 2008, the receiver 2010 mentioned above, an open retainer 2012 and the insert 2014 with friction fit collet 2015, also mentioned above. The assembly 2001 is shown with a closure structure 2018 and also with a 5.5 mm diameter rod 2021 that is identical or substantially similar to the rod 21 previously described herein. The assembly 2001 may be used with a 6.0 mm diameter rod, similar to the rod 21′ previously described herein, as well as other types of longitudinal connecting members. The shank 2004 is identical or substantially similar to the shank 4 previously described herein and thus includes a spherical surface 2036 terminating at a top rim surface 2038, the upper portion surface 2036 having a hemisphere 2040 and also a drive feature 2046 formed therein that are the same or substantially similar in form and function to the respective spherical surface 36, top surface 38, hemisphere 40 and drive feature 46 previously described herein with respect to the shank 4.

The receiver 2010 also includes a variety of features that are the same as or substantially similar to the features of the receiver 10 previously described herein. Thus, the receiver 2010 includes a base 2060, surfaces defining an inner cavity 2061, a pair of opposed arms 2062 forming a U-shaped channel 2064 that has an opening 2066 and also communicates with the cavity 2061 and opposed inner arm surfaces 2070 having flange form guide and advancement structures 2072 terminating near top surfaces 2073 that are identical or substantially similar in form and function to the respective base 60, surfaces defining the inner cavity 61, pair of opposed arms 62 forming the U-shaped channel 64, the channel opening 66 that communicates with the cavity 61 and opposed inner arm surfaces 70 having flange form guide and advancement structures 72 terminating near top surfaces 73 of the arms previously described herein with respect to the receiver 10.

Although the receiver 2010 also includes outer arm surfaces 2076 that further include shallow tool receiving recesses or apertures 2077 that are the same or substantially similar in form and function to the respective receiver 10 arm surfaces 76 and recesses 77 previously described herein, the receiver 2010 differs from the receiver 10 in that formed below each aperture or recess 2077 is a through aperture or bore, generally 2079 formed in and through each of the outer surfaces 2076. Each aperture 2079 has a generally up-side down U-shape, the U-shape aperture defining a central inwardly and upwardly extending holding tab 2080 integral with the respective arm 2062 at or near the base 2060 and generally extending upwardly from the receiver base 2060 and inwardly toward a receiver central axis B. Each aperture 2079 extends through the respective arm surface 2076 to the respective inner arm surface 2070. Each aperture 2079 is located spaced from the adjacent aperture 2077 and near or adjacent the receiver base 2060.

The assembly 2001 is typically provided to a user with the insert 2014 being held within the receiver 2010 by the pair of inwardly extending holding tabs 2080, that are typically somewhat resilient, firmly holding the insert 2014 during assembly with the shank 2004 and keeping the insert 2014 relatively stationary with respect to the receiver 2010 in an upward position between the arms 2062 until the insert 2014 is pressed into movable friction fit with the shank upper portion or head 2008. The holding tabs 2080 advantageously hold the insert 2014 in a centered position (the insert arms being held in alignment with the receiver arms) during rotation and torquing of the closure top 2018 onto the rod 2021 or other connecting member. The opposed holding tabs 2080 include outer surfaces and also various inner surfaces for contacting the insert 2014. The tab surfaces include a first outer surface 2081 extending from the base 2060 and sloping upwardly and slightly inwardly toward the receiver axis B. A tab top surface 2082 is substantially perpendicular to the surface 2081, the top surface 2082 running toward the axis B and terminating at an inner surface 2084. The inner surface 2084 slopes downwardly and inwardly from the top surface 2082 and terminates at another inwardly facing surface 2085 that terminates at a lower lip or bottom surface 2086. The inner surfaces 2085 and 2086 and the bottom surface 2086 are sized and shaped for engaging the insert 2014 as will be described in greater detail below. In some embodiments of the invention the inner surfaces 2085 and 2086 may be combined to form a single surface that may be slightly concave or cylindrical and may be substantially perpendicular to the top surface 2082. In the illustrated embodiment, the surface 2084 is frusto-conical, but may be cylindrical or planar in other embodiments. The illustrated lower inner surface 2086 is cylindrical and is disposed substantially perpendicular to the bottom lip 2086. Located adjacent to the bottom lip 2086 and extending downwardly is a transition surface 2088 that angles toward and transitions into a cylindrical surface 2094 that defines a substantial portion of the receiver inn cavity 2061 and is otherwise substantially similar to the surfaces 94 and 98 previously described herein with respect to the receiver 10. Because the insert 2014 does not include resilient outwardly extending portions like the insert 14, the receiver 2010 does not include recessed portions of greater diameter such as the surfaces 92 and 96 of the receiver 10. However, all of the other surfaces defining the cavity 2061 located below the surface 2094 are substantially similar in form and function to the surfaces previously described herein that define the cavity 61 of the receiver 10 and shall not be further described herein other than to identify a seating surface 2103 and a receiver lower opening 2110 that are the same or substantially similar to the respective seating surface 103 and opening 110 of the receiver 10. The lower or bottom tab surface 2088 is parallel to the top surface 2082. The holding tabs 2080 are stable, but exhibit some resilience, being pushed outwardly away from the axis B during rotation of the insert 2014 when the insert 2014 is being assembled with the receiver 2010 as shown, for example, in FIG. 68. Each holding tab 2080 further includes opposed side surfaces 2089 that partially define the U-shaped portion of the through aperture 2079. The aperture 2079 is further defined by a top surface 2090 and opposed outer substantially planar side surfaces 2091, each surface 2091 being spaced from and opposed to a tab surface 2089 with both the surfaces 2091 and 2089 terminating at curved bottom surfaces 2092.

Returning to the interior surface 2070 of the receiver arms 2062, a discontinuous cylindrical surface 2093 having a diameter slightly less than a diameter of the lower cylindrical surface 2094 is located below the guide and advancement structure 2072 and above the surface 2094. It is noted that more or fewer surfaces of different diameters may be provided between the guide and advancement structure 2072 and the surface 2094 in order to closely receive the insert 2014 during assembly of the insert into the receiver 2010 and also during subsequent operation of the overall assembly 2001 to capture and fix the rod 2021 within the receiver 2010.

The retainer 2012 is identical or substantially similar in form and function to the retainer 12 previously described herein with respect to the assembly 1. Thus, the retainer 2012 includes an outer cylindrical surface 2120, a bottom surface 2122, a top surface 2126, grooves or notches 2128, a radiused inner surface 2143, an inner frusto-conical surface 2145, a slit 2149 and other similar features that are the same or substantially similar to the outer cylindrical surface 120, bottom surface 122, top surface 126, grooves or notches 128, radiused inner surface 143, inner frusto-conical surface 145, slit 149 and other features of the retainer 12 previously described herein.

The insert 2014 includes numerous features that are the same or substantially similar to the insert 14 previously described herein with respect to the assembly 1. Thus, the insert 2014 includes an upper body 2156, a pair of opposed arms 2157 with top surfaces 2160, the collet 2015 with bottom surfaces 2162, a through bore 2180, a rod receiving saddle surface 2182, an inner cylindrical surface 2185 and an inner radiused surface 2188 having slits or grooves 2189 that are substantially similar in form and function to the respective upper body 156, pair of opposed arms 157 with top surfaces 160, collet 15 with bottom surfaces 162, through bore 180, rod receiving saddle surface 182, inner cylindrical surface 185 and inner radiused surface 188 having slits or grooves 189 of the insert 14 previously described herein. However, the illustrated insert 2014 does not include the shank gripping portion 190 of the insert 14. Rather, the radiused lower surfaces 2188 are smooth and extend upwardly to and terminate at the inner cylindrical surface 2185.

Also, unlike the insert 14, the arms 2157 of the insert 2014 do not include edm cuts, slots or recesses that create outer resilient portions, but rather the insert 2014 has shallow grooves and apertures formed in the arm outer surfaces for receiving and engaging the resilient tabs 2080 of the receiver 2010. These features include: an outer cylindrical surface 2166 that runs from the top surface 2160 to an outer band or raised surface 2167 that is also cylindrical and has a diameter slightly greater than a diameter of the cylindrical surface 2166. The outer band surface 2167 is evenly spaced from the collet bottom 2162 and runs across top portions of the slits 2189 in a direction perpendicular to the slits 2189. Frusto conical or curved surfaces transition between the outer cylindrical surface 2166 and the outer band 2167. Formed centrally in each surface 2166 between the top surface 2160 and the band 2167 is a shallow recess or aperture 2168 defined by a base surfaces 2169 and a perimeter wall 2170 having a substantially rectangular profile. The wall 2170 extends outwardly from the base 2169 to the arm cylindrical outer surface 2166. The aperture 2168 ultimately captures a respective receiver tab 2080 as will be described in greater detail below. Running from directly below the aperture 2168 and also formed in the surface 2166 is a trough or groove 2172, sized and shaped to receive and slidingly engage one of the receiver resilient tabs 2080 at the surfaces 2084, 2085 and 2086 during assembly of the insert 2014 with the receiver 2010 when the insert 2014 is rotated into place, as shown, for example, in FIG. 68. The trough 2172 terminates at an end surface or stop 2173 that is located directly below the perimeter wall 2170 of the recess 2168. Near the bottom 2162 of the collet portion 2015 and below the outer band 2167 is a cylindrical surface 2178 that has a diameter that is the same as the diameter of the outer surface 2166.

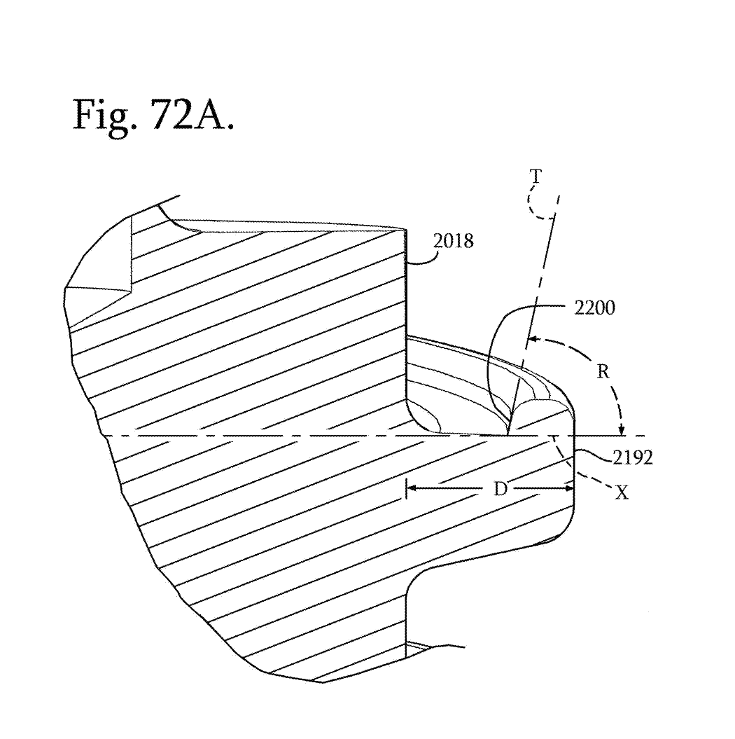

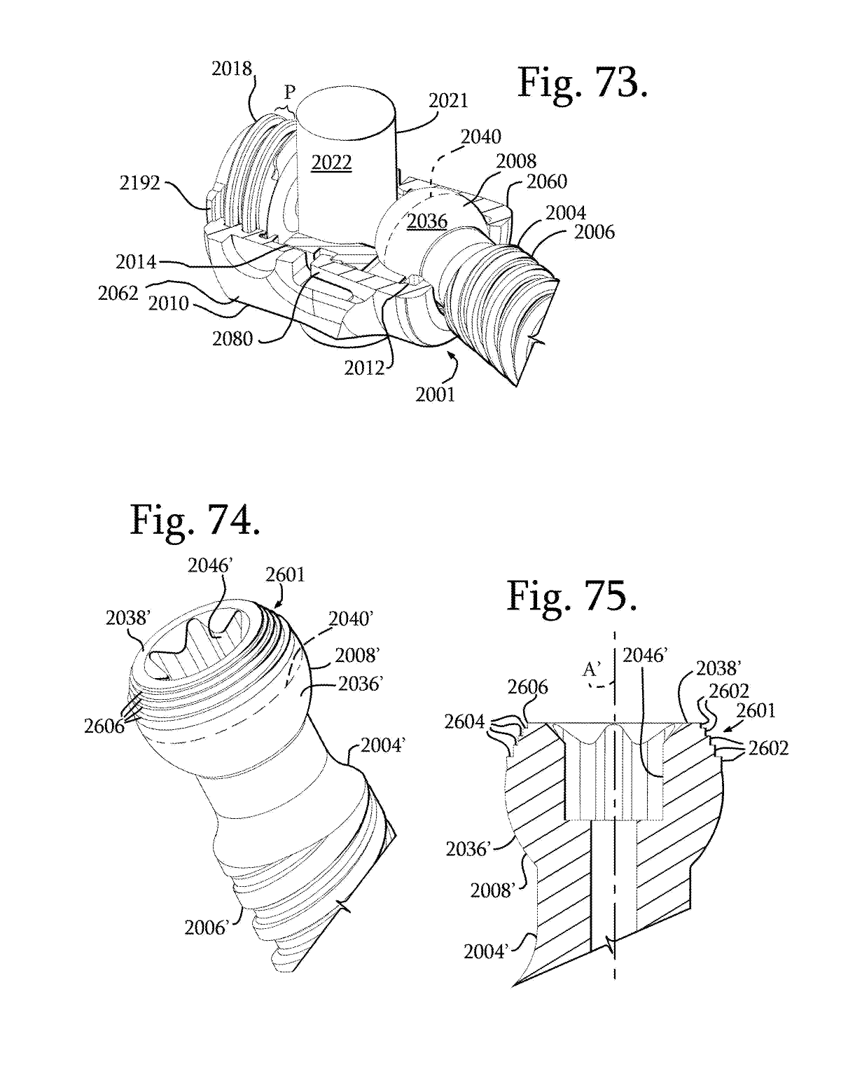

With reference to FIGS. 72, 72A and 73, the closure structure 2018 is substantially similar in form and function to the structure 18 previously described herein. Thus, the structure 2018 includes a flange form structure 2192, an internal drive 2196, a base or bottom surface 2197, a bottom nub 2198 and an annular mound around the nub 2199 that are substantially similar in form and function to the respective flange form structure 192, internal drive 196, base or bottom surface 197, bottom nub 198 annular mound around the nub 199 previously described herein with respect to the closure 18. Because the structure 2018 is only shown in a final stage of assembly with the receiver 2010, a break-off head is not shown. It is noted that closures 2018 may be provided with or without break-off heads and may include other geometry at the base 2197 in lieu of the nub 2198 and annular portion 2199 that are illustrated herein. Furthermore, with particular reference to FIG. 72A, it is noted that the illustrated flange form structure 2192 is a dual start structure that has a flange form depth D measured from a root to a crest of the flange form 2192 of between about 0.7 and about 0.8 millimeters. The flange form structure 2192 further has a pitch P (axial distance between flange forms, for example, as shown in FIG. 73 from a particular crest point or location to a next crest point or location) of about 0.100 inches. Returning to FIG. 72A, the flange form structure 2192 also has a loading flank surface 2200 (shown extended as a line T in phantom) that is disposed at an angle R of about eighty degrees with respect to a radius or reference line X perpendicular to a central axis of the closure 2018. It is noted that with such a geometry, particularly with such a large pitch, a desirable material for the closure structure 2018 is cobalt chrome so as to counter possible loosening that may occur under cyclical loading. If the structure 2018 is made from cobalt chrome, a desirable material for the cooperating receiver 2010 is titanium or a titanium alloy.

With particular reference to FIGS. 67-71, the receiver 2010, retainer 2012 and compression insert 2014 are typically assembled at a factory setting that includes tooling for holding and alignment of the component pieces and manipulating the retainer 2012 and the insert 2014 with respect to the receiver 2010. In some circumstances, the shank 2004 is also assembled with the receiver 2010, the retainer 2012 and the compression insert 2014 at the factory. In other instances, it is desirable to first implant the shank 2004, followed by addition of the pre-assembled receiver, retainer and compression insert at the insertion point. In this way, the surgeon may advantageously and more easily implant and manipulate the shanks 2004, distract or compress the vertebrae with the shanks 2004 and work around the shank upper portions or heads 2008 without the cooperating receivers 2010 being in the way. In other instances, it is desirable for the surgical staff to pre-assemble a shank 2004 of a desired size and/or variety with the receiver 2010, retainer 2012 and compression insert 2014. Allowing the surgeon to choose the appropriately sized or treated shank 2004 (or any other compatible shank, such as one with a uni-planar pivot range) advantageously reduces inventory requirements, thus reducing overall cost and improving logistics and distribution.

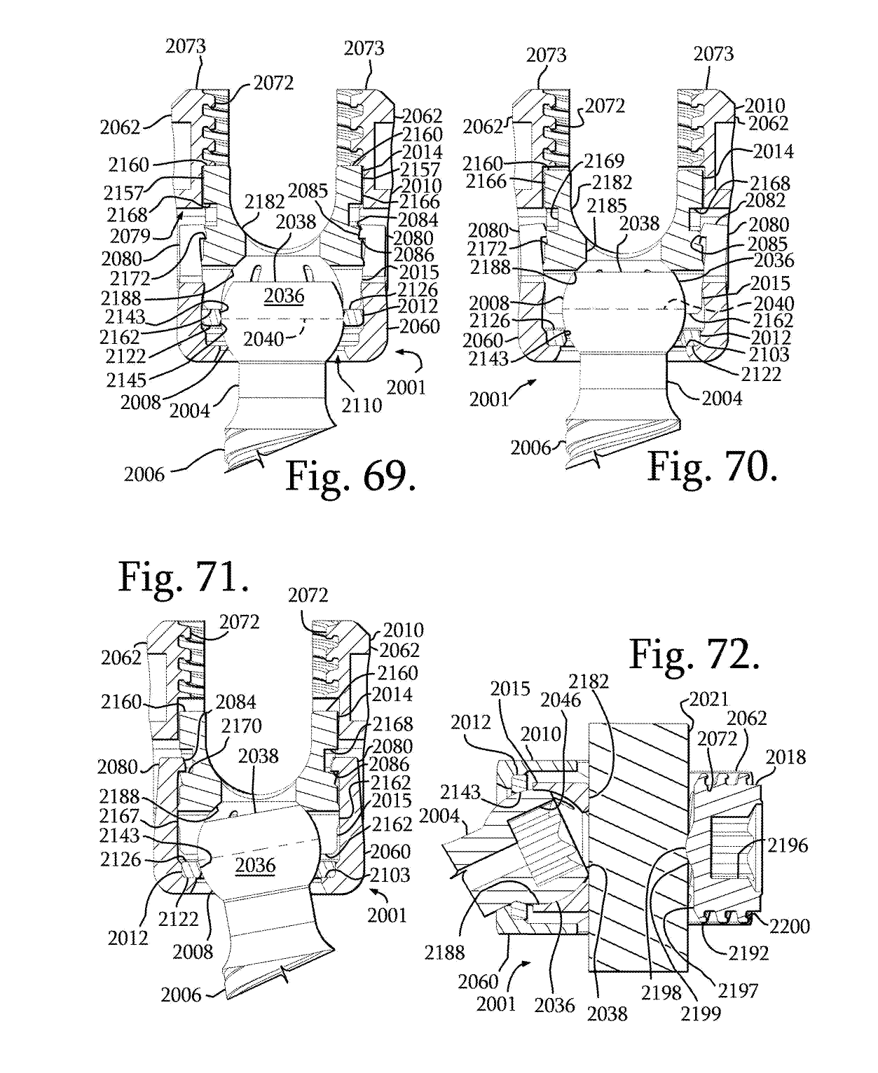

With particular reference to FIG. 67, first the retainer 2012 is inserted into the upper receiver opening 2066, followed by the insert 2014 in a manner as previously described herein with respect to the assembly of the retainer 12 and insert 14 in the receiver 10. At this time, the retainer 2012 is free to rotate with respect to the receiver about the axis B. The compression insert 2014 is downloaded into the receiver 2010 through the upper opening 2066 with the bottom surface 2162 facing the receiver arm top surfaces 2073 and the insert arms 2157 located between the opposed receiver arms 2062. The insert 2014 is then lowered toward the receiver and between the arms 2062 with the insert body 2156 initially in a tight or press fit arrangement with the receiver 2010 at the guide and advancement structures 2072 located on the inner surfaces 2070 near the top surfaces 2073 of the arms 2062. Force is used to move the insert body 2156 between the guide and advancement structures 2072, slightly splaying the arms 2062 away from one another. The receiver 2010 is preferably made from a resilient material such as a stainless steel or titanium alloy, to allow for a temporary outward splaying of the arms 2062 during initial insertion of the insert 2014. Also, a preferred material for the insert 2014 is a cobalt-chrome alloy that is harder than a material of the receiver 2010. With reference to FIG. 68, as soon as the body 2156 of the insert 2014 clears the guide and advancement structures 2072 and is situated within the receiver arm and receiver cylindrical surfaces 2093 and 2094, the resilient receiver arms 2062 return to an original orientation and the insert 2014 is now captured within the receiver 2010 also capturing the retainer 2012 within the receiver 2010 below the insert and above the seating surface 2103. The insert 2014 is then lowered to a position wherein the insert 2014 arm top surfaces 2160 are adjacent to run-out areas below each of the receiver arm guide and advancement structures 2072. Thereafter, the insert 2014 is rotated about the receiver axis B as shown in FIG. 68 until each upper arm surface 2160 is directly below one of the guide and advancement structures 2072 as shown in FIG. 69. During the rotation step, portions of the receiver resilient tabs 2080, namely the surfaces 2084, 2085 and 2086 slide in one of the insert troughs 2172 until the receiver tab abuts against the insert stop surface 2173. At such time, each of the resilient tabs 2080 is located directly beneath one of the insert apertures 2168 and the insert 2014 is desirably aligned with the receiver 2010 with the insert arms 2157 aligned with the receiver arms 2062. The insert 2014 is now captured in a desired shipping position wherein the guide and advancement structures 2072 of the receiver 2010 prohibit upward movement of the insert 2014 and the receiver tab 2080 portions located within the insert grooves 2172 prohibit downward movement of the insert 2014. The receiver 2010, retainer 2012 and insert 2014 combination is now in a desired pre-assembled state and ready for assembly with the shank 2004 either at the factory, by surgery staff prior to implantation, or directly upon an implanted shank 2004 as will be described herein.

The bone screw shank 2004 or an entire assembly 2001 made up of the assembled shank 2004, receiver 2010, retainer 2012 and compression insert 2014, is screwed into a bone, such as the vertebra 17 as described previously with respect to the assembly 1. With reference to FIGS. 69-71, whether it is desired for the shank 2004 to be “popped” on to the receiver pre-assembly (receiver 2010, retainer 2012 and insert 2014) before or after implantation of the shank 2004 into bone, the following steps apply: With reference to FIG. 69, the receiver 2010 is placed over the shank head 2008 top surface 2038 and the shank is “popped” into the receiver by pushing the shank head 2008 into the receiver opening 2110 and the shank surface 2036 into contact with the retainer frusto-conical surface 2145. The retainer 2012 and shank head 2008 are then moved further into the receiver cavity 2061 defined by the cylindrical surface 2094 with the shank head hemisphere 2040 clearing the receiver opening 2110. The shank head 2008 then pushes the retainer top surface 2126 into abutment with the insert bottom surface 2162. With reference to FIG. 69, as the shank head 2008 continues to push upwardly, the retainer 2012 begins to expand outwardly toward the receiver cylindrical surface 2094. FIG. 69 shows a maximum expansion of the retainer 2012 about the shank head 2008 with upward movement of the retainer 2012 being blocked by the insert 2014 that in turn is blocked from upward movement by the insert top surfaces 2160 abutting against the receiver guide and advancement structures 2072. FIG. 69 also illustrates the shank head 2008 pushing the slitted insert collet 2015 outwardly as well. FIG. 70 illustrates full capture of the shank head 2008 by the retainer 2012 with the retainer 2012 dropping to the bottom seating surface 2103 of the receiver 2010. The hemisphere 2040 of the shank head 2008 is now located entirely above the retainer 2012. Also, the insert collet 2015 inner surface 2188 is now in full frictional engagement with the shank surface 2036 located above the hemisphere 2040. However, at this time, the shank head 2008 and insert 2014 are still held in an upper portion of the receiver cavity 2061 by the receiver resilient tabs 2080 pressing against the insert 2014 at the surfaces of the grooves 2172 as well as the insert 2014 outer band surface 2167 being in very close or some frictional engagement with inner surfaces 2094 located at or near the tabs 2080, requiring a pulling up of the receiver 2010 away from an implanted shank 2004, for example, or a pulling down of the shank 2004 away from the receiver 2010 to urge the insert 2014 downwardly into a desired position wherein the receiver resilient tabs 2080 slip or snap or otherwise deploy into the opposed insert recesses 2168 with the surfaces 2085 and 2086 located within the aperture or recess 2168 but being spaced from the back surface 2169 that partially defines the aperture or recess 2168. As shown in FIG. 71, after such a pulling away of the receiver 2010 from the shank 2004, the tabs 2080 resiliently return to a neutral or near neutral position with the receiver tab bottom surfaces 2086 engaging lower or bottom portions of the recess perimeter wall 2170. Also, when each tab 2080 return to a neutral position, a lower portion or portions of the tab body moves away from the insert surface 2167. With further reference to FIG. 71, the insert is now captured in a desired position within the receiver 2010 by the receiver spring tabs 2080 as well as a close but movable fit between the insert outer band surface 2167 and portions of the receiver surface 2094 located at and/or near the spring tabs 2080. At this time the insert collet surfaces 2188 remain in frictional or friction fit engagement with the shank head surface 2036 while allowing pivoting of the shank 2004 with respect to the receiver 2010 when some force is applied to the shank 2004 or to the receiver 2010 to place the shank and receiver into a desired angular orientation with one another, for example, as shown in FIG. 71. The shank and receiver may be placed in a variety of angular orientations with respect to one another, using some force, and such orientation will be maintained by the friction fit relationship between the shank head 2008 and the collet 2015 portion of the insert 2014. Thus, desirable, non-floppy angular adjustments may be made during surgery. Also any undesirable rotational movement of the insert 2014 with respect to the receiver 2010 is prohibited by the vertical wall portions (running parallel to the axis B) of the perimeter wall 2170 that define the insert recess 2168 that are now located adjacent each of the side walls 2089 of the receiver resilient tabs 2080. Slight rotational movements result in the side walls 2089 abutting against the adjacent perimeter wall 2170.

With reference to FIGS. 72 and 73, the assembly 2001 as shown in FIG. 71 is further shown being assembled with a 5.5 millimeter rod 2021 and the closure top 2018 previously described herein. The closure top 2018 is driven into the receiver guide and advancement structure 2072 using a socket type driver (not shown) that receives the break-off head (not shown). As the driver is rotated, the closure top 2018 guide and advancement structure 2192 is fully mated with the receiver guide and advancement structure 2072 causing downward movement of the closure top 2018 onto the rod 2021, the rod in turn pressing downwardly on the insert 2014, pressing the insert deeper into the receiver 2010 and frictionally fixing or locking the insert 2014 against the shank head 2008 which is now in a fixed position and no longer pivotable with respect to the receiver 2010. The angle of the shank 2004 with respect to the receiver 2010 is the same in FIGS. 71 and 72, with the shank being at about a twenty-five degree angle with respect to the receiver. FIG. 73 shows a different orientation of the shank 2004 with respect to the receiver 2010 wherein an angle of pivot or inclinations is also about twenty-five degrees, but in a medial direction.

It is noted that if the surgeon wishes to further manipulate the rod 2021 or remove the rod, the closure top 2018 may be loosened (and removed of desired) by using a driver in the closure drive 2196 to rotate the closure 2018 and move the closure 2018 in an upward direction away from the rod 2021. At such time, the receiver 2010 can again be tilted or otherwise angularly manipulated with respect to the shank 4 in a friction fit movable, but non-floppy manner using some force.

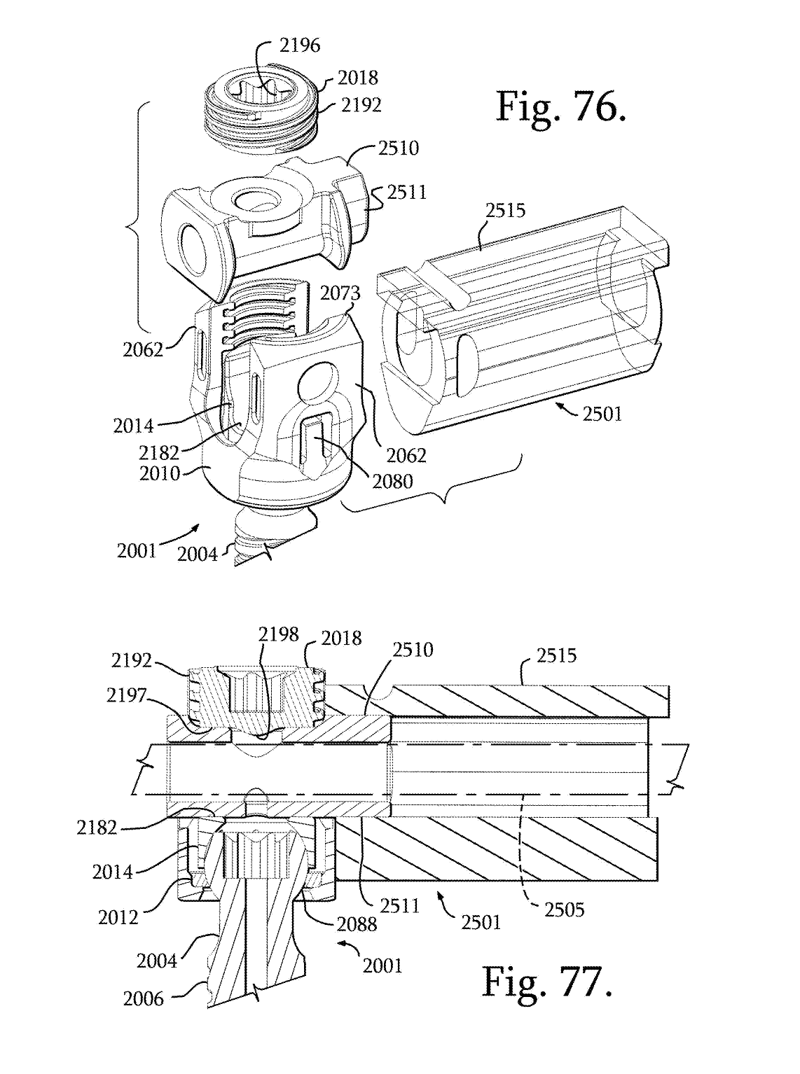

Similar to the assembly 1 shown in FIGS. 33 and 34, the closure 2018 with lower nub 2198 advantageously cooperates with rods or other longitudinal connecting members having various diameters. Furthermore, with respect to FIGS. 76 and 77, the assembly 2001 is shown with a portion of a soft or dynamic longitudinal connecting member assembly, generally 2501, that includes a tensioned cord 2505, a rigid sleeve 2510, and a spacer 2515. The assembly portion 2501 is the same or substantially similar in form and function to soft stabilization assemblies described in Applicant's U.S. patent application Ser. No. 13/573,516 that has already been incorporated by reference herein, the rigid sleeve 2510 having an inner through bore for slidingly receiving the tensioned cord 2505 and being sized and shaped to be received by the insert 2014 at the saddle surface 2182. The sleeve 2510 may be made from a variety of materials, preferably hard materials, including, but not limited to stainless steel, titanium and titanium alloys and cobalt chrome. The sleeve further includes at least one extended or protruding portion 2511 that extends into a through bore of the spacer 2515. The spacer 2510 may be made from hard or soft materials and thus may be compressible. The illustrated spacer 2510 is shown being made of a transparent plastic material. As shown in FIG. 77, the closure 2018 presses down on the sleeve 2510 that in turn presses the insert 2014 into fixed frictional engagement with the shank head 2008 that in turn presses against the retainer 2012 that is pressed both downwardly and outwardly against the receiver 2010. The illustrated closure 2018 bottom nub 2198 remains spaced from the tensioned cord 2505 and thus the cord is free to slip or slide with respect to the sleeve 2510 and thus with respect to the bone screw assembly 2001. An alternative closure (not shown) includes an extended portion or point for fixing the cord 2505 with respect to the sleeve 2510, if desired, and is more fully described in Applicant's '516 patent application.

With reference to FIGS. 74 and 75, an alternative bone screw shank 2004′ having a shank body 2006′ and an integral upper portion or head 2008′ is illustrated that may be used in the assembly 2001 in lieu of the shank 2004. The shank 2004′ may also be used in other bone screw embodiments described herein. The shank 2004′ is identical to the shank 2004 with the exception of graduated surface tiers, generally 2601, formed into a shank head 2008′ outer spherical surface 2036′ above a shank head hemisphere 2040′. The tiers 2601 are made up of alternating cylindrical surfaces 2602 and planar annular surfaces 2604 that are perpendicular to one another and define circular edges 2606 that generally follow a radius that is the same or close to a radius of the surface 2036′. The cylindrical surfaces are coaxial with a central axis A′ of the shank 2004′. In the illustrated embodiment, there are five cylindrical surfaces 2602 and four annular surfaces 2604. However more or fewer surfaces may be cut into the shank head surface 2036′ and may include other surfaces sizes and other geometric shapes. The illustrated cylindrical surfaces 2602 begin near the shank hemisphere 2040′ with an upper smallest and shortest surface 2602 terminating at an edge 2606 that also defines a termination of a top surface 2038′ of the shank 2004′. As the surfaces 2602 advance upwardly toward the top surface 2038′, they become shorter in height and lesser in diameter. Similarly, each of the planar annular surfaces 2604 is more narrow than an annular surface 2604 located directly there below. In operation, when the bone screw 2001 is in an ultimate fixed frictional relation to a rod or other longitudinal connecting member, the edges 2606 engage and preferably penetrate or dig in to the insert 2014 lower spherical surface 2188. Such a digging in advantageously occurs when the shank 2004 is made from a harder material than a material of the insert 2014′. For example, the shank 2004 may be made from cobalt chrome and the insert 2014 from stainless steel or titanium or titanium alloy.

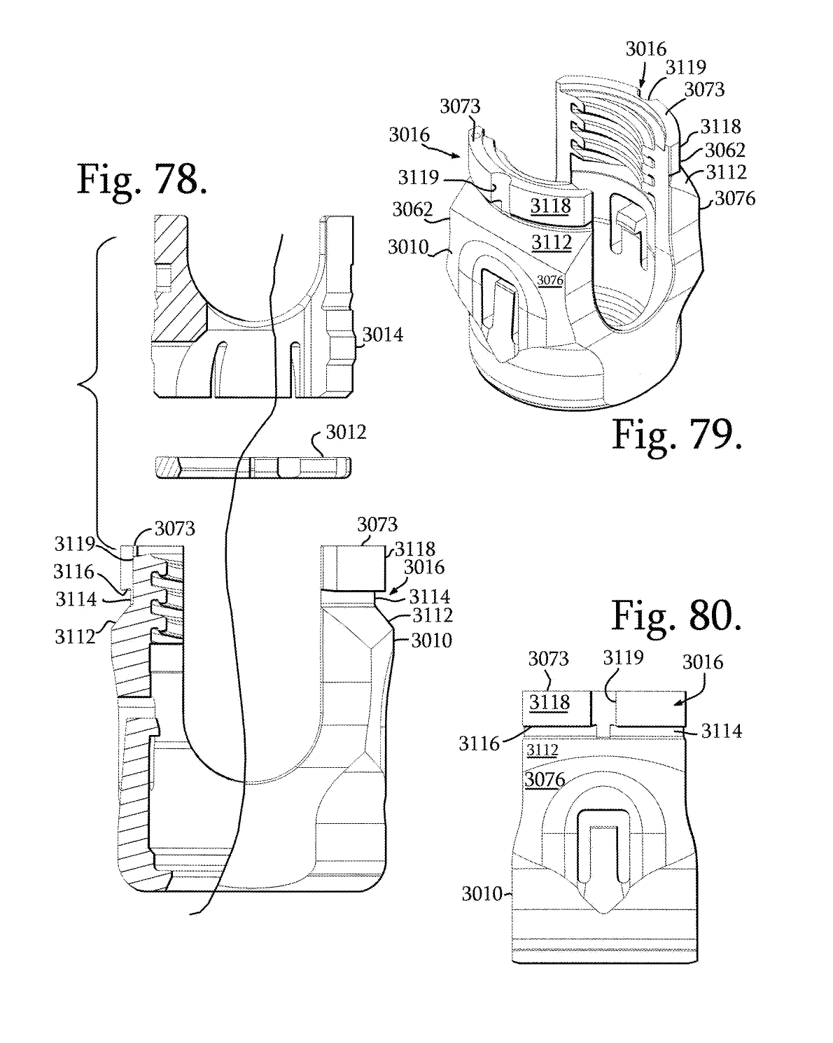

With reference to FIGS. 78-83, an alternative bone screw assembly, generally 3001 is shown that includes a shank 3004 having a threaded body 3006 and an integral upper portion or head 3008, a receiver 3010, an open retainer 3012 and an insert 3014. The bone screw assembly is substantially similar to the bone screw assembly 2001 previously discussed herein. The only feature that distinguishes the assembly 3001 from the assembly 2001 is an upper tool engaging structure, generally, 3016, located on each of the arms of the receiver 3010. Rather than having opposed arms 2062 with outer arm surfaces 2076 that extend almost all the way to arm top surfaces 2073, the receiver 3010 has the tool engaging structure 3016 located on each arm between the arm outer surfaces 3076 and arm top surfaces 3073. Specifically, the receiver tool engaging structure 3016 on each arm 3062 includes an inwardly and upwardly sloping surface 3112 extending from the surface 3076 to a curved or partially cylindrical neck 3114. The neck 3114 extends upwardly to an outwardly extending planar lip 3116, the lip 3116 being substantially perpendicular to the neck 3114 or positioned at an angle with respect to the neck of slightly less than ninety degrees. Extending upwardly from the lip 3116 is another curved or partially cylindrical surface portion 3118 that extends to the top surface 3073. The neck surfaces 3114 and the upper outer cylindrical surfaces each have a radius that originates at a central axis of the receiver 3010, the upper cylindrical surface 3118 radius being greater than the neck 3114 radius. The illustrated lip 3116 is slightly undercut from the upper cylindrical surface 3118 to the neck 3114. Carved centrally in each upper cylindrical surface 3118 is a vertical slot or groove 3119 that extends through the top surface 3073 and the lower lip 3116. The illustrated groove 3119 also extends partially into the neck surface 3114. Thus there are two grooves 3119 that are opposed to one another and run parallel to the central axis of the receiver 2010. The grooves 3119 are located and sized and shaped for receiving tooling (not shown). Although the illustrated grooves 3119 have curved surfaces, in other embodiments some or all of the surfaces defining the grooves may be planar.

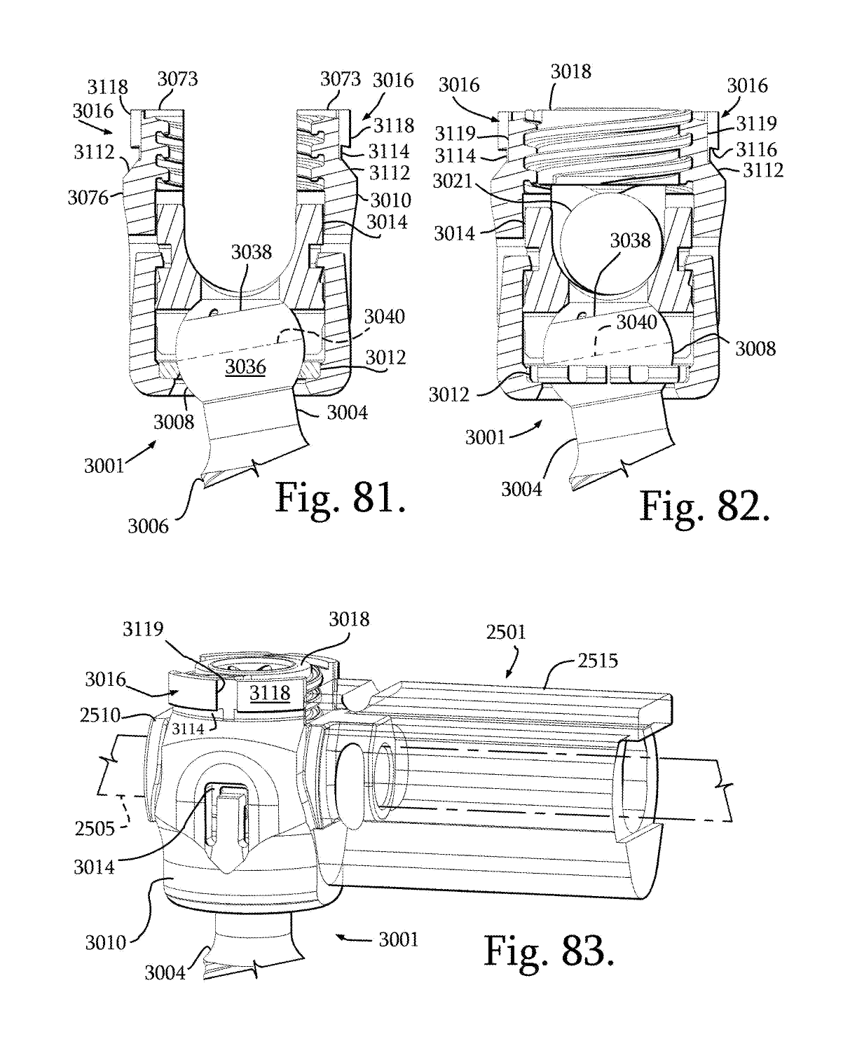

As stated above, the assembly 3001 otherwise includes all of the structure and features previously described herein with respect to the assembly 2001 and thus the receiver 3010, retainer 3012 and the insert 3014 will not be described in detail herein. It is noted that certain features may be sized slightly differently in order to accommodate the tool receiving structure 3016 on the receiver 3010. However, the assembly 3010 otherwise is assembled and functions in a manner identical to what has been described previously herein with respect to the assembly 2001. The assembly 3001 components are shown fully assembled in FIG. 81 and further shown in fixed relation with a rod 3021 and closure 3018 in FIG. 82. The closure 3018 is identical to the closure 2018 previously described herein. With reference to FIG. 83, the assembly 3001 is shown with the soft stabilization assembly portion 2501 previously described herein with respect to the assembly 2001 that includes the tensioned cord 2505, rigid sleeve 2510 and spacer 2515.

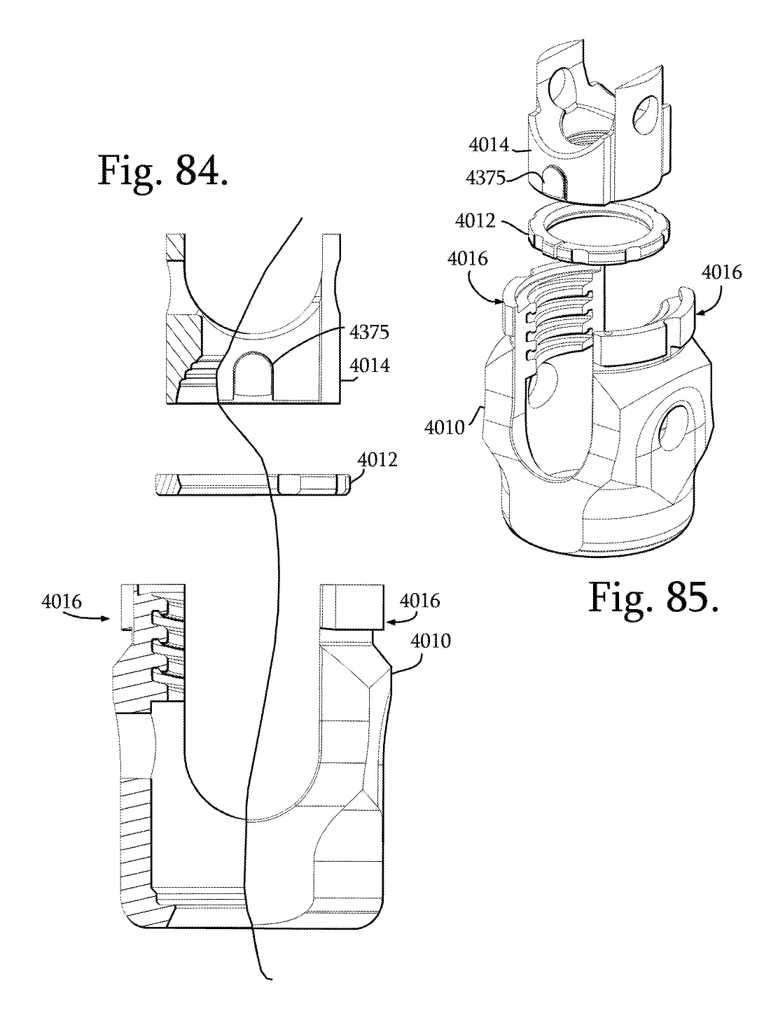

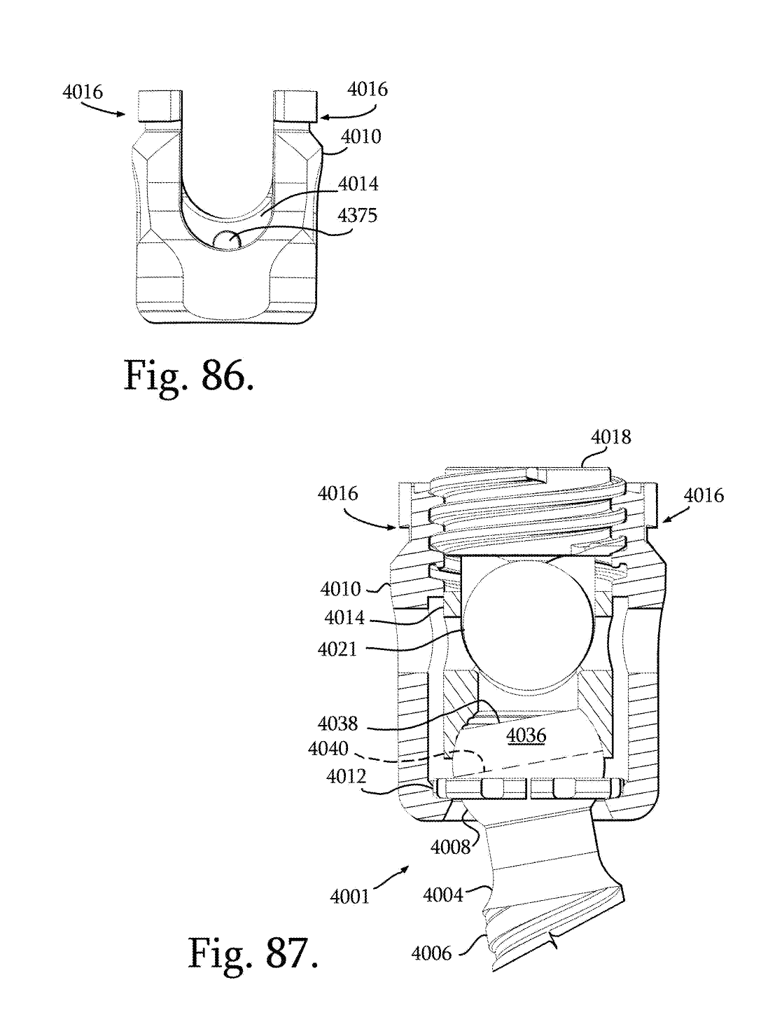

With reference to FIGS. 84-87 an alternative bone screw assembly, generally 4001 is shown that includes a shank 4004 having a threaded body 4006 and an integral upper portion or head 4008, a receiver 4010, an open retainer 4012 and an insert 4014. The bone screw assembly is substantially similar to the bone screw assembly 201 shown in FIGS. 37-56 and previously described herein having the identical or substantially similar respective bone screw shank 204, receiver 210, retainer 212 and insert 214. The only feature that distinguishes the assembly 4001 from the assembly 201 are upper tool engaging structures on each receiver arm, generally, 4016. Each of the tool engaging structures 4016 is identical or substantially similar to the tool engaging structures 3016 previously described herein located on the arms of the receiver 3010 of the assembly 3001 and thus shall not be described further. All of the other features of the receiver 4010 are identical or substantially similar to the features of the receiver 210 previously described herein. Also, the open retainer 4012 and the insert 4014 are identical or substantially similar to the retainer 212 and the insert 214 previously described herein. Like the insert 214 with strips 375, the insert 4014 includes outer structures or strips 4375 for advantageously engaging the receiver 4010 in a diametric friction fit engagement as previously described herein with respect to the assembly 201. The components of the assembly 4001 may be assembled in a manner identical to what has been described herein with respect to the assembly 201. With reference to FIG. 87, the assembly 4001 is shown assembled with a larger rod 4021 (6.0 mm diameter) and with a closure 4018 that is the same or substantially similar to the closure 2018 previously described herein.

It is to be understood that while certain forms of the present invention have been illustrated and described herein, it is not to be limited to the specific forms or arrangement of parts described and shown.

What is claimed and desired to be secured by Letters Patent is as follows:

1. In a medical implant, the improvement comprising:
a) a bone anchor shank with a body for fixation to a bone and an integral upper portion having a curved first surface with a first radius and a hemisphere;
b) a bone anchor receiver having a base and a pair of upright arms forming an open channel, the channel partially defined by arm inner surfaces for receiving a closure, the base defining a chamber and having a lower opening, the channel communicating with the chamber, the receiver base having a central axis and a loading surface located near the lower opening;
c) an open retainer captured within the chamber and expandable about the shank upper portion, the retainer having a bottom surface sized and shaped for engagement with the receiver loading surface, the retainer being positioned entirely below the shank upper portion hemisphere when in a final locked position with respect to the shank and the receiver with the retainer bottom surface frictionally engaging the receiver loading surface, the retainer frictionally engaging the shank curved surface when in the final locked position, the retainer being in expansion-only locking engagement with both the shank upper portion and the receiver;
d) an insert located within the receiver, the insert having an inner lower surface in a friction fit with the shank upper portion during temporary manipulation of the shank with respect to the receiver, the shank upper portion being movable with respect to the insert with some resistance when a force is applied to the shank to pivot the shank with respect to the receiver, the insert being in locked frictional engagement with the shank upper portion when a force is applied on the insert that in turn presses the retainer into the expansion only locked position; and wherein
e) the retainer is made from a first material and the receiver is made from a second material, the first material being harder than the second material.

2. The improvement of claim 1 wherein the first material is a cobalt-chrome alloy and the retainer has one and up to a plurality of grooves formed in one of an inner and outer surface thereof.

3. The improvement of claim 1 wherein the second material is a titanium alloy.

4. The improvement of claim 1 wherein the retainer and the insert are made from a first material and the receiver and the shank are made from a second material, the first material being harder than the second material.

5. The improvement of claim 4 wherein the closure is made from the first material.

6. The improvement of claim 1 wherein the shank body has two thread forms at a bottom thereof and three thread forms near the upper portion.

7. The improvement of claim 1 wherein the insert has diametrically opposed outer surfaces in frictional engagement with inner front and rear surfaces of the receiver spaced from the receiver arms.

8. The improvement of claim 1 wherein the insert has a pair of opposed arms, each arm having a slot running downwardly from a top surface of the respective arm, the slot forming a resilient outer arm portion that is spaced from and pressable toward a remainder of the arm.

9. The improvement of claim 8 wherein the insert has a pair of opposed projections located on outside surfaces of the resilient outer arms portions and each receiver arm has a ledge for receiving one of the outer projections.

10. The improvement of claim 1 wherein the insert has a pair of opposed arms, each arm having an outer surface with a shallow aperture formed therein and the receiver has opposed resilient arm tabs frictionally engaging the insert at the shallow apertures.

11. The improvement of claim 10 wherein each insert arm further has a groove located below each shallow aperture, each receiver arm tab sliding along one of the grooves during assembly of the insert with the receiver.

12. The improvement of claim 11 wherein the receiver arm tabs are located in the grooves during assembly of the receiver with the shank.

13. The improvement of claim 1 wherein the shank upper portion has a first planar surface adjacent the curved surface and wherein the insert has a second planar surface, the first and second planar surfaces being adjacent and limiting pivoting of the shank to a single plane.

14. The improvement of claim 13 wherein the retainer has a third planar surface adjacent the shank upper portion first planar surface.

15. The improvement of claim 1 wherein the receiver has opposed resilient arm tabs frictionally engaging the insert.

16. A bone anchor comprising:
a) a shank with a body for fixation to a bone and an integral upper portion having a curved first surface with a first radius and a hemisphere;
b) a receiver having a base and a pair of upright arms forming an open channel, the channel partially defined by arm inner surfaces for receiving a closure, the base having inner front and rear surfaces located between the receiver arms, the base defining a chamber and having a lower opening, the channel communicating with the chamber, the receiver base having a central axis and an inner seating surface located near the lower opening;
c) an open retainer captured within the chamber and expandable about the shank upper portion, the retainer having a bottom surface sized and shaped for engagement with the receiver seating surface, the retainer being positioned entirely below the shank upper portion hemisphere when in a final locked position with respect to the shank and the receiver with the retainer bottom surface frictionally engaging the receiver seating surface, the retainer frictionally engaging the shank curved surface when in the final locked position, the retainer being in expansion-only locking engagement with both the shank upper portion and the receiver; and
d) an insert located within the receiver, the insert having an inner lower surface in frictional engagement with the shank upper portion, the insert having diametrically opposed outer surface portions in frictional engagement with the receiver inner front and rear surfaces.

17. The bone anchor of claim 16 wherein the retainer and the insert are made from a first material and the shank and the receiver are made from a second material, the first material being harder than the second material.

18. The bone anchor of claim 16 wherein the retainer and the shank are made from a first material and the insert and the receiver are made from a second material, the first material being harder than the second material.

19. The bone anchor of claim 16 wherein the shank body has one and up to a plurality of thread forms near a bottom thereof and greater than two thread forms near the upper portion.

20. The bone anchor of claim 16 wherein the shank body has two thread forms near a bottom thereof and three thread forms near the upper portion.

21. The bone anchor of claim 16 wherein the shank upper portion has a first planar surface adjacent the curved surface and wherein the insert has a second planar surface, the first and second planar surfaces being adjacent and limiting pivoting of the shank to a single plane.

22. The bone anchor of claim 21 wherein the retainer has a third planar surface adjacent the shank upper portion first planar surface.

23. In a pedicle screw having a lower threaded shank for insertion into bone and a head, the improvement comprising a shank body having a first threaded portion having one and up to a plurality of thread forms, a second threaded portion with more than two thread forms, and a non-interleaved transition portion located between the first and second portions, the transition portion including both the first portion thread forms and the second portion thread forms and wherein a minor diameter of the shank remains substantially constant at the transition portion and a first major diameter measured at the transition portion is one of equal to and less than a second major diameter of the first portion and a third major diameter of the second portion.

24. The improvement of claim 23 wherein the second portion is an upper portion of the shank located between the transition portion and the head.

25. The improvement of claim 23 wherein the first portion has two thread forms and the second portion has three thread forms.
